(19) United States
(12) Patent Application Publication
(10) Pub. No.: US 2014/0370080 A1
(43) Pub. Date: Dec. 18, 2014

(54) HISTONE DEACETYLASE (HDAC) INHIBITORS FOR TREATMENT OF POST-SURGICAL ADHESIONS

(71) Applicant: TRUSTEES OF BOSTON UNIVERSITY, Boston, MA (US)

(72) Inventors: Arthur F. Stucchi, Southborough, MA (US); Michael Cassidy, Boston, MA (US)

(21) Appl. No.: 14/351,994

(22) PCT Filed: Oct. 26, 2012
(86) PCT No.: PCT/US2012/062176

§ 371 Date: Apr. 15, 2014

Related U.S. Application Data
(60) Provisional application No. 61/552,063, filed on Oct. 27, 2011.

(51) Int. Cl. A61K 31/4406; A61K 31/167; A61K 45/06; A61K 31/19

(52) U.S. Cl. CPC A61K 31/4406; A61K 31/19; A61K 31/167; A61K 45/06
USPC 424/450; 514/557; 514/616; 514/357; 424/158.1

(57) ABSTRACT

The present invention relates generally to methods, compounds and compositions for treatment of adhesions, such as surgical adhesions.

### RELATED APPLICATIONS

This application claims benefit under 35 U.S.C. §119(e) of the U.S. Provisional Application No. 61/552,063, filed Oct. 27, 2011, the content of which is incorporated herein by reference in its entirety.

### TECHNICAL FIELD

The present disclosure relates generally to methods, compounds, and compositions for treatment and/or reduction of surgical adhesions.

### BACKGROUND

Nearly 100% of people who undergo an abdominal or pelvic operation will develop internal scars called intraabdominal adhesions. Adhesions form between internal organs and the intestines, the consequences of which can eventually cause a blockage in the intestine, infertility in women, chronic pelvic pain and high risk re-operative procedures. Intestinal blockages can be life threatening and while most are managed with medical therapy, surgery is often required. The currently approved technology to prevent or reduce adhesions are in a class called physical barriers, which are basically biodegradable sheets of film, like waxpaper in consistency, that are placed over suspected sites of scar formation. These solid sheets are not only difficult to use in all surgical applications, but they tend to only be effective where they are placed and have no remote effects due to the lack of a soluble drug. Further, physical barriers are not applicable in all surgeries and only act locally. There are no currently available pharmaceutical agents that can decrease postoperative adhesions.

Thus, there is need in the art for methods, compounds, and compositions for treating or reducing the formation of adhesions after surgery

### SUMMARY

Provided herein are novel methods, compounds and compositions for reducing, inhibiting, preventing or treating the formation of surgical adhesions. These methods are based, in part, on inventors' discovery that histone deacetylase (HDAC) inhibitors are unexpectedly and surprisingly effective in reducing the incidence of adhesions. By way of experimentation, applicants have shown that at least three different HDAC inhibitors (Valproic acid, SAHA, and MS-275) are effective in reducing adhesion formation in a post-surgical adhesion model. Accordingly, in one aspect, provided herein is a method of reducing, inhibiting, preventing or treating adhesion formation, the method comprising administering to a subject in need thereof a therapeutically effective amount of an HDAC inhibitor, a derivative thereof or a prodrug thereof.

The method described herein can be used to treat post-surgical adhesion(s). Thus, in some embodiments, the method comprises administering to a subject an effective amount of a HDAC inhibitor, derivative thereof or a prodrug thereof to thereby treat a surgical adhesion of said subject.

In some embodiments, the adhesion is intra-abdominal or peritoneal adhesion.

In some embodiments, the adhesion is a post-surgical adhesion.

In some embodiments, the HDAC inhibitor is valproic acid, SAHA, and MS-275, or any combination thereof.

The HDAC inhibitor can be administered pre-surgically (i.e. pre-operatively), during surgery (i.e. intraoperatively), or post-surgically (i.e. post-operatively). Accordingly, in some embodiments, the HDAC inhibitor can be administered in a pre-operative dose. In some other embodiments, the HDAC inhibitor can be administered in an intra-operative dose. In yet some other embodiments, the HDAC inhibitor can be administered in a post-operative dose. In still some other embodiments, the HDAC inhibitor can be administered in any combination of two or more of a pre-operative dose, intra-operative dose and post-operative dose, for example by a pre-operative and intra-operative dose. In some embodiments, the HDAC inhibitor can be administered in a single intra-operative dose.

In some embodiments, the HDAC inhibitor can be administered parenterally. In some embodiments, the HDAC inhibitor can be administered intraperitoneally.

Further, the HDAC inhibitor can be administered in any suitable form. For example, the HDAC inhibitors can formulated be in a solution (e.g. a soluble form); in a gel; in a spray; in a controlled-dose or controlled release formulation; or incorporated into a physical barrier (e.g., a biodegradable barrier) that is placed in the subject during surgery. The HDAC inhibitor can be administered in a mixture for formulation. The HDAC inhibitors can be administered in various sustained release gels and polymers. The HDAC inhibitor can also be formulated into a composition further comprising a pharmaceutically acceptable carrier.

In some embodiments, the HDAC inhibitor can be formulated into a liposome or micelle for administration to the subject.

In some embodiments, the HDAC inhibitor can be formulated into a gel for administration to the subject.

In some embodiments, the HDAC inhibitor can be applied to a biodegradable barrier that can be placed in the subject during surgery.

In some embodiments, the method comprises contacting a subject with an HDAC inhibitor during surgery wherein the contacting is performed by way of an intraperitoneal injection.

In some embodiments, the method comprises contacting a subject with an HDAC inhibitor during surgery wherein the contacting is performed by placing a barrier comprising a HDAC inhibitor in the subject.

In some embodiments, the subject is a mammal. In some embodiments, the subject is a human.

Further provided herein is the use of a HDAC inhibitor for the manufacture of a medicament for treatment of surgical adhesions. In some embodiments, the medicament can further comprise at least one other therapeutic agent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings, described below, are for illustration purposes only. The drawings are not intended to limit the scope of the present teaching in any way. Further, in the drawings, the sizes and relative positions of elements in the drawings are not necessarily drawn to scale. For example, the shapes of various elements and angles may not be drawn to scale, and some of these elements are arbitrarily enlarged and positioned to improve drawing legibility. Further, the particular shapes of the elements as drawn may not be intended to convey any information regarding the actual shape of the particular elements, and may have been selected solely for ease of recognition in the drawings.

FIG. 1A is a bar graph showing adhesion formation (in percent) in rats for a control group and for those rats to which was administered intraperitoneally three (3) doses (24 hours pre-operative, intra-operative and 24 hours post-operative) of valproic acid (in either 200 mg/kg or 100 mg/kg). The data shows that both dosing regimens were effective to reduce the severity of surgical adhesions but that the lower dose of 100 mg/kg was more effective.

FIG. 1B is a bar graph showing adhesion formation (in percent) in rats for a control group and for those rats to which was administered intraperitoneally three (3) doses (24 hours pre-operative, intra-operative and 24 hours post-operative) of valproic acid (in either 100 mg/kg or 50 mg/kg) The data shows that both dosing regimens were effective to reduce the severity of surgical adhesions but that the lower dose of 50 mg/kg was more effective.

FIG. 2A is a bar graph showing adhesion formation (in percent) in rats for a control group and for those rats to which was administered 50 mg/kg intraperitoneally in a single intra-operative dose as compared with three doses (pre-op, intra-op and pre-op) of 25 mg/kg of valproic acid as a HDAC inhibitor. The data shows that both dosing regimens were effective. However, the single intra-operative dose of 50 mg/kg was superior to multiples doses of 25 mg/kg.

FIG. 2B is a bar graph showing adhesion formation (in percent) in rats for a control group and for those rats to which was administered 50 mg/kg intraperitoneally in a single intra-operative dose as compared with three (3) doses (24 hours pre-operative, intra-operative and 24 hours post-operative) of 50 mg/kg of valproic acid as a HDAC inhibitor. The data suggests that both dosing regimens are about equally effective. A single intra-operative dose can be preferable for its simplicity.

FIG. 3A is a bar graph showing adhesion formation (in percent of control) in rats for a control group and for those rats to which was administered three doses (pre-operation, intra-operation and post-operation) of various amounts (200 mg/kg to 25 mg/kg) of valproic acid as a HDAC inhibitor. The data shows that all dosing regimens were effective but that the single intra-operative dose of 50 mg/kg was the most effective regimen.

FIG. 3B is a bar graph showing data for burst pressure in rats for a control group and for those rats to which was administered 50 mg/kg intraperitoneally in a single intra-operative dose. This data illustrates that there were no negative effects cause by the treatment with the HDAC inhibitor under the conditions tested.

FIG. 4 is a bar graph showing adhesion formation (in percent) in rats for a control group and; (i) for those rats to which was administered 50 mg/kg intraperitoneally in a single intra-operative dose of SAHA and 10 mg/kg of MS-275 as a HDAC inhibitor. The data shows that both HADC inhibitors SAHA (also called Verinostat) and MS-275 (also called Entinostat) were also effective for the treatment of surgical adhesions when administered in a single intra-operative dose.

### DETAILED DESCRIPTION

Provided herein are novel methods for inhibiting, reducing, and/or treating adhesion formation or adhesiogenesis. These methods are based on the inventors' discovery that HDAC inhibitors are unexpectedly and surprisingly effective in reducing the incidence of adhesions. Please note that any reference to a HDAC inhibitor herein includes pharmaceutically acceptable salts thereof, derivatives thereof, and prodrugs thereof.

In one aspect provided herein is a method for the treatment and inhibition of adhesions and adhesion formation, the method comprising administering to a subject an effective amount of a HDAC inhibitor, a derivative thereof or a prodrug thereof to thereby treat a surgical adhesion of said subject. Accordingly, the method described herein pertains to treating a disease state in a subject by administering to said subject: (i) an HDAC inhibitor; (ii) compositions, mixtures or formulations comprising said HDAC inhibitors; (iii) prodrugs of said HDAC inhibitors; (iv) derivatives of HDAC inhibitors; or iv) combination therapies using said HDAC inhibitors, said compositions, said prodrugs and/or said derivatives.

Without limitations, the method described herein can be used as a method for treating, preventing, or minimizing implant adhesions such those resulting from natural or synthetic, autologous or heterologous, implants used in breast, abdominal wall, cosmetic, orthopedic, craniofacial, cardiac, or urologic surgeries. Accordingly, the method described herein can be used in the treatment of post-operative adhesions, adhesions resulting from trauma, or adhesions resulting from a fibrotic disease. Further, the treatment can be prophylactic or to prevent an increase in the severity of an existing adhesion

The method described herein is particularly useful in the treatment and inhibition of adhesions formed in the peritoneal or pelvic cavities as a result of a wound, surgery, infection, inflammation, or trauma. The method described herein has been shown to be especially effective in preventing adhesion formation in the peritoneum following surgery. In addition, the method described herein find utility in other contexts, e.g., for cardiovascular, orthopedic, thoracic, ophthalmic, CNS and other uses, where prevention of the formation of adhesions is a significant concern and complication following surgery at these sites.

Accordingly, the methods described herein are useful for the treatment of, inhibition of, suppression of, or reduction of the formation or reformation of adhesions, resulting from wound, surgery, infection, inflammation, trauma, or any combination thereof. Treatment or inhibition of adhesions can include, but is not limited to, a lowering or decrease in the incidence of adhesions, a decrease in the size of adhesions, or a decrease in the rate of adhesiogenesis.

Efficacy of the methods of treatment described herein can be assessed, for example by measuring a marker, indicator, symptom or incidence of adhesions as described herein or any other measurable parameter appropriate, e.g. intestinal blockage, female infertility and chronic pelvic pain. The incidence of adhesions can also be examined by, for example laparoscopy or laparotomy. It is well within the ability of one skilled in the art to monitor efficacy of treatment or prevention by measuring any one of such parameters, or any combination of parameters.

The terms “treat,” “treatment,” “treating,” or “amelioration” refer to therapeutic treatments for adhesion formation, wherein the object is to reverse, alleviate, ameliorate, inhibit, slow down or stop the progression or severity of a symptom or condition associated with adhesion formation. The terms “treat,” “treatment,” “treating,” and “amelioration” include reducing or alleviating at least one adverse effect or symptom of a condition, disease or disorder associated with adhesiogenesis. Treatment is generally “effective” if one or more symptoms or clinical markers are reduced. Alternatively, treatment is “effective” if the progression of a disorder is reduced or halted. That is, “treatment” includes not just the improvement of symptoms or markers of adhesion, but also a cessation of at least slowing of progress or worsening of symptoms of adhesion that would be expected in absence of treatment. Beneficial or desired clinical results include, but are not limited to, alleviation of one or more symptom(s), diminishment of extent of the disorder, stabilized (i.e., not worsening) state of the disorder, delay or slowing of disorder progression, amelioration or palliation of the disorder state, and remission (whether partial or total), whether detectable or undetectable. The term “treatment” of a disorder also includes providing relief from the symptoms or side-effects of the disorder (including palliative treatment).

By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the terms “prevent,” “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the complete avoidance or prevention of symptoms, but also a reduced severity or degree of any one of those symptoms, relative to those symptoms arising in a control or non-treated individual (e.g. a normally healthy subject) with a similar likelihood or susceptibility of developing the disease or disorder, or relative to symptoms likely to arise based on historical or statistical measures of populations affected by the disease or disorder.

Effective treatment is evident when there is a statistically significant improvement in one or more markers, indicators, or symptoms of adhesion or adhesiogenesis, or by a failure to worsen or to develop symptoms where they would otherwise be anticipated. As an example, a favorable change of at least 10% in a measurable parameter of adhesion or adhesiogenesis, and preferably at least 20%, at least 30%, at least 40%, at least 50%, at least 60%, least 70%, at least 80%, at least 90%, at least 95%, at least 99%, or more can be indicative of effective treatment. Efficacy for a given HDAC inhibitor or a composition comprising the same can also be judged using an experimental animal model known in the art for a condition described herein. When using an experimental animal model, efficacy of treatment is evidenced when a statistically significant change in a marker is observed, e.g. the incidence of adhesion formation, size of adhesion, rate of adhesion formation.

In some embodiments, administration of a HDAC inhibitor according to the method described herein can reduce levels of a marker or symptom of adhesions or the incidence, size, or rate of formation of adhesions by at least 10%, at least 15%, at least 20%, at least 25%, at least 30%, at least 40%, at least 50%, at least 60%, at least 70%, at least 80% or at least 90%, at least 95%, at least 98%, at least 99%, or up to and including a 100% reduction, i.e., no adhesion formation.

After surgery or trauma results in injury to a tissue, e.g., the peritoneum, via ischemia, electrocautery, or abrasion, there is a rapid influx of inflammatory cells into the tissue, e.g., peritoneum, which elicits an acute inflammatory response and initiates a cascade of events intended to facilitate normal wound healing. The inflammatory response can cause a fibrinous exudate to form between adjacent organs. As inflammatory cells infiltrate the fibrinous exudate, the material becomes organized, eventually forming dense bands of fibrous tissue connecting a tissue to an organ, i.e., adhesions.

Intra-abdominal or peritoneal adhesions occur in more than 94% of patients after abdominal surgery and also arise after pelvic surgeries. Adhesions can result in intestinal blockage, female infertility, chronic pelvic pain and high risk re-operative procedures. Adhesions are also problematic in orthopedic and plastic surgeries, such as in the hand, where impediment of movement is frequently troublesome to the patient.

Accordingly, the term “adhesion,” as used herein in the medical sense, refers to a conglutination, an adhering or uniting of two surfaces or parts, such as between two organ surfaces. For example, the union of the opposing surfaces of a wound, or opposing surfaces of peritoneum. Also, adhesions, in the plural, can refer to bands of connective tissue that connect opposing serous surfaces. The term adhesion, as used herein, also includes “fibrinous adhesions,” which are adhesions that comprise fine threads of fibrin resulting from an exudate of plasma or lymph, or an extravasion of blood. “Keloid,” which refers to a smooth overgrowth of fibroblastic tissue that arises in an area of injury or, occasionally, spontaneously is also a form of adhesion. “Basal adhesion formation,” as used herein in its medical sense, is the basal level of adhesion formation that occurs after injury wounding (e.g. surgery) exposed to an atmosphere which contains sufficient oxygen to avoid a condition of hypoxia or of hyperoxia. The method described herein is useful in the treatment of all such adhesions.

In some embodiments, an adhesion to be treated or inhibited using the methods described herein is one that forms at or occurs between organ surfaces. As used herein, the term “organ surface” is intended to encompass any internal organ or tissue of a living animal including, but not limited to the uterus, intestine, peritoneum, omentum, stomach, liver, kidneys, heart, and lung

As used herein, the term “trauma” or “traumatic injury” is intended to encompass any wound, insult, or noxious stimulus to a membrane, or tissue or organ surface. In some embodiments, the traumatic injury can be caused by a surgical procedure. In some other embodiments, the traumatic injury is not caused by a surgical procedure.

As used herein, the term “abdominal adhesions” or “peritoneal adhesions” refer to the bands of fibrous tissue that cause abdominal organs to adhere to one another or to the abdominal wall.

As used herein, adhesions or adhesion formation or adhesion reformation that arises during or following a surgical procedure, are termed herein as a “post-operative adhesion” or a “surgical adhesion.” Postoperative adhesion formation or adhesions are a major complication that occur during or following, for example, a general or plastic surgical procedure including, but not limited to, abdominal, gynecologic, cardiothoracic, spinal, plastic, vascular, ENT, ophthalmologic, urologic, neuro, or orthopedic surgery. Surgical adhesions are typically connective tissue structures that form between adjacent injured areas within the body.

In some embodiments, the methods provided herein are useful for the treatment or inhibition of “adhesiogenesis.” The terms “adhesiogenesis,” “adhesion formation” or “newly formed adhesions,” as used herein in the medical sense, refer to conglutination, or the process of adhering or uniting of two surfaces or parts that were not previously thus united or adhered. For example, the union or adhering of the opposing surfaces of a wound, opposing surfaces of peritoneum, or between the surface of the peritoneum and another tissue or organ.

While surgical traumas (e.g., incisions, suturing, etc) are a major cause of adhesion formation, any trauma is capable of inducing adhesion formation. Accordingly, in some embodiments, the methods described herein are directed to the inhibition, amelioration, or treatment of adhesions caused by trauma. In some embodiments, the methods described herein are directed to the inhibition, amelioration, or treatment of adhesions caused by a non-surgical trauma.

As used herein, the term “trauma” or “traumatic injury,” is intended to encompass any wound, insult, or noxious stimulus to a membrane or tissue surface. Traumas that can be treated by embodiments of the methods described herein can be a result of a breach of the membrane or tissue boundary. In other embodiments, the traumas that can be treated are not a result of a breach of the membrane or tissue boundary. Traumas can result from a disease condition, for example, vascular insufficiency or infection associated with a pathogen, burns (thermal or chemical), or from application of external force to a membrane or tissue surface by accident or surgery. Noxious stimuli include the action of heat or corrosive chemicals, for example acids and caustics, as well as manipulation of an organ during surgery.

Non-surgical trauma can include, but is not limited to, inflammation, infection, burns, ischemia, blunt force trauma, incisions, repetitive stress trauma, crush injuries, and contact with a hard surface. Accordingly, in some embodiments, the methods described herein comprise administering a HDAC inhibitor to a trauma site.

In some embodiments, the traumatic injury can be caused by a surgical procedure. In some other embodiments, the traumatic injury is not caused by a surgical procedure.

Adhesions or adhesion formation can arise following a surgical procedure, termed herein as a “post-operative adhesion” or a “surgical adhesion.” Postoperative adhesion formation or adhesions are a major complication that occur following, for example, a general or plastic surgical procedure including abdominal, gynecologic, cardiothoracic, spinal, plastic, vascular, ENT, ophthalmologic, urologic, neuro, or orthopedic surgery. Surgical adhesions are typically connective tissue structures that form between adjacent injured areas within the body. Briefly, localized areas of injury trigger an inflammatory and healing response that culminates in healing and scar tissue formation. If scarring results in the formation of fibrous tissue bands or adherence of adjacent anatomical structures (that should be separate), surgical adhesion formation is said to have occurred. Adhesions can range from filmy, relatively easily separable structures to dense, tenacious fibrous structures that can only be separated by surgical dissection. While many adhesions are benign, some can cause significant clinical problems and are a leading cause of repeat surgical intervention. Surgery to breakdown adhesions (adhesiolysis) often results in failure and recurrence because the surgical trauma involved in breaking down the adhesion triggers the entire process to repeat itself.

Postoperative adhesions generally occur as a result of a normal wound healing response within the fifth to seventh day after injury. It is considered that adhesion formation and adhesion-free re-epithelialization are alternative pathways, both of which begin with coagulation, and which initiate a cascade of events resulting in the buildup of fibrin gel matrix. In the case where fibrin deposition is in excess or not removed, the fibrins crosslink to form the fibrin gel matrix, which may then serve as a progenitor or scaffolding on which to form adhesions. The crosslinked scaffold rapidly becomes infiltrated with cellular elements, such as fibroblasts, which produce extracellular matrix materials, such as collagen, which provides the basis for adhesion formation. In contrast to this, a protective system for fibrinolytic enzyme in peritoneum, such as the plasmin system, can remove the fibrin gel matrix. However, surgery and surgical procedures dramatically attenuate fibrinolytic activity. Therefore, it is determined depending upon the extent of the damage and fibrinolysis in the tissue surface which pathway of adhesion formation and re-epitheliazation is selected.

Accordingly, in some embodiments, the methods described herein can be used for the treatment, inhibition, amelioration, or reduction of post-operative adhesions formed during a surgical procedure, i.e., adhesion formation caused by surgery. Examples of surgeries that can cause adhesion formation include, but are not limited to, cesarean section, laparoscopic surgery, abdominal surgery, pelvic surgery, cardiac surgery, pulmonary surgery, ocular surgery, otologic surgery, orthopedic surgery, breast surgery, intestinal surgery, peritoneal surgery, bladder surgery, gynecological surgery, spine surgery, rectal surgery, dental surgery, laparoscopic surgery and various kinds of plastic surgery.

While adhesions in the abdomen can develop as after-effects of peritonitis, or due to abdominal trauma, mostly, and particularly in developed countries, such adhesions most commonly result from abdominal surgical procedures during which organs are traumatized by surgical manipulations. Abdominal or peritoneal adhesions refer to the bands of fibrous tissue that cause abdominal organs to adhere to one another or to the abdominal wall. Common, non-limiting examples of abdominal adhesions include intestine-to-intestine, and intestine-to-pelvic organs, intestine-to-abdominal wall, and omentum to any of these sites. In some such cases, abdominal adhesion formation can have severe effects and complications. Postoperative peritoneal adhesion is often accompanied with complications such as intestinal obstruction, infertility, and pain in the serious cases after abdominal or gynecologic surgery (Dig. Surg. 18: 260-73, 2001, content of which is incorporated herein by reference in its entirety). For example, in some individuals, constricting adhesions block the flow of contents through the intestines, a condition called intestinal obstruction. In certain instances, a segment of bowel becomes twisted around an adhesive band, thus cutting off the normal blood supply. The affected portion of the intestine becomes non-viable and can perforate. Accordingly, in some embodiments, a subject being treated using the methods described herein is having or has had an abdominal surgical procedure or suffers from abdominal adhesions.

Furthermore, once abdominal adhesions have formed, they do not resolve. Their lysis, by operation, only temporarily eliminates them. For example, when surgery is performed for adhesive intestinal obstruction caused by adhesions, adhesions routinely re-form and later cause a new intestinal obstruction in 11%-21% of such cases. In some embodiments, a subject being treated using the methods described herein has previously had a surgery to treat or remove an abdominal adhesion.

Adhesions can also form as a result of surgery to or in the pelvic area. Adhesions formed in the pelvic area due to surgical procedures, such as, for example, cesarean sections, can lead to severe complications, such as female infertility. Accordingly, in some embodiments, a subject being treated using the methods described herein is having or had had a surgery in the pelvic region. In some such embodiments, the subject is a female subject.

Pericardial adhesions, which refer to adhesions formed within the pericardium of the heart, pose a significant problem and increase morbidity and mortality of reoperative cardiac surgical procedures (J. Invest. Surg. 14: 93-97, 2001, content of which is incorporated herein by reference in its entirety). Accordingly, in some embodiments, a subject being treated using the methods described herein is having or had a cardiac surgery or suffers from pericardial adhesions.

Also provided herein are methods of treating, inhibiting, ameliorating, or minimizing post-surgical adhesions between an implant and surrounding tissues at a surgical site, such as implants used in breast, cardiac, cosmetic, orthopedic (e.g., hand, knee, hip, foot), craniofacial, or urologic surgeries. Implants used in surgical procedures include, but are not limited to, grafting material, transplanted organs, and medical devices. Tissues surrounding implants used in surgical procedures include, but are not limited to, fascia, soft tissues, muscle, organs, fat, adipose, membranes, pericardium, plura, periostium, peritoneum, dura, bowels, intestines, ovaries, veins, arteries, epidermis, tendons, ligaments, nerves, bone and cartilage.

A grafting material can comprise autologous or autograft material, heterologous materiel, i.e., xenograft material or allograft material, or any combination thereof. Examples of grafting material include veins, arteries, heart valves, skin, dermis, epidermis, nerves, tendons, ligaments, bone, bone marrow, blood, white blood cells, red blood cells, gonadocytes, embryos, cells, adipose, fat, muscle, cartilage, fascia, membranes, matrix materials including artificial and/or naturally-occurring components such as, for example, collagen and/or other tissues or components such as, but not limited to, connective tissues, matrix materials including biological or naturally-occurring matrix materials and/or including artificial materials, polymers formed partially or entirely of biological or naturally-occurring materials such as, for example, collagen and/or other tissues or components such as, but not limited to, connective tissues and/or artificial materials, pericardium, plura, periostium, peritoneum, and dura. Implants can include a transplanted organ, such as kidneys, hearts, eyes, and livers, among other things.

Implants comprising non-biological materials, and/or medical devices, include, but are not limited to, bone graft substitutes, bone cement, tissue glues and adhesives, bone fixation members, defibrillators, eye spheres, sutures, staples, cochlear implants, pumps, artificial organs, non-resorbable membranes, bone growth stimulators, neurological stimulators, dental implants, guided tissue and guided bone regeneration membranes, eyelid weights, and tympanostomy tubes. Other such implants can include prosthetics, such as a fluid filled prosthesis. One example of a fluid filled prosthesis is a breast implant, such as a saline or silicone breast implant. In addition, medical devices can include electronic instruments, such as a pacemaker.

Accordingly, the methods described herein can, in some embodiments, be used to attenuate, minimize, reduce, or inhibit adhesions formed between an implant or foreign body and surrounding tissues following a surgical procedure, such as a coronary surgical procedure. In such embodiments, the surrounding tissue can comprise bone, soft tissues, muscle, organs, membranes, pericardium, veins and arteries, and the implant can comprise, for example, one or more of (a) grafting materials which can comprise one or more of veins, arteries, heart valves, muscle, membranes, matrix materials including artificial and/or naturally-occurring components such as, for example, collagen and/or other tissues or components such as, but not limited to, connective tissues, matrix materials including biological or naturally-occurring matrix materials and/or including artificial materials, polymers formed partially or entirely of biological or naturally-occurring materials such as, for example, collagen and/or other tissues or components such as, but not limited to, connective tissues and/or artificial materials, and pericardium, (b) coronary tissue comprising one or more of a heart, veins, arteries, heart valves, muscle, membranes, matrix materials including biological matrix materials, polymers formed partially or entirely of biological materials such as collagen, and pericardium, and (c) medical devices which can comprise one or more of tissue glues and adhesives, sutures, staples, defibrillators, pacemakers, pumps, artificial organs or parts or components thereof, non-resorbable or partially non-resorbable membranes, and guided tissue regeneration membranes.

In some embodiments, the methods described herein are directed to the treatment, reduction, or inhibition of adhesions caused by fibrotic disorders. Fibrotic disorders are characterized by the abnormal formation of an extracellular matrix and can lead to the development of adhesions. Non-limiting examples of fibrotic disorders for use with the methods described herein include endometriosis, pulmonary fibrosis, systemic sclerosis, scleroderma, proliferative vitreoretinopathy, hepatic cirrhosis and mesangial proliferative cell disorders. Further examples of fibrotic disorders include cystic fibrosis of the pancreas and lungs, endomyocardial fibrosis, idiopathic pulmonary fibrosis of the lung, mediastinal fibrosis, myelofibrosis, retroperitoneal fibrosis, nephrogenic systemic fibrosis, progressive massive fibrosis (a complication of coal workers' pneumoconiosis) and injection fibrosis (which can occur as a complication of intramuscular injections, especially in children). Further fibrosis-associated conditions include diffuse parenchymal lung disease, post-vasectomy pain syndrome, tuberculosis (which can cause fibrosis of the lungs), sickle-cell anemia (which can cause fibrosis of the spleen) and rheumatoid arthritis.

Fibrosis has certain similarities to adhesiogenesis, although the processes are distinct in causative origins. By way of a non-limiting example, adhesiogenesis can be caused by trauma and/or surgery, while, for example, idiopathic pulmonary fibrosis and hepatic fibrosis are caused, respectively, by foreign bodies in the lung or hepatitis. Additionally, the molecular pathways that govern the two phenomenons can differ.

In another aspect, provided herein are inhibitors of adhesion, for use in methods of treating, inhibiting, minimizing, or ameliorating adhesion formation or reformation in subjects in need thereof. As used herein, an “inhibitor of adhesion,” or “adhesion inhibitor” refers to a compound or agent that inhibits, reduces, or treats adhesion formation or reformation of adhesions, resulting from wound, surgery, infection, inflammation, trauma, or any combination thereof. An inhibitor of adhesion is any agent that can treat or inhibit adhesion formation or reformation, and includes any agent that lowers or decreases the incidence of adhesions, decreases the severity of adhesions, decreases the size of adhesions, inhibits the rate of adhesiogenesis, or any combination thereof. In some embodiments, the adhesion inhibitor is an HDAC inhibitor, a derivative thereof, a prodrug thereof, or a pharmaceutically acceptable salt thereof. In some embodiments, the adhesion inhibitor is used for the treatment of post-surgical or post-operative adhesions.

As used herein, an “HDAC inhibitor” means a compound that has the ability to inhibit or interfere with function or activity of a histone deacetylase. More specifically, to carry out gene expression, a cell must control the coiling and uncoiling of DNA around histones. This is accomplished with the assistance of histone acetylases (HAT), which acetylate the lysine residues in core histones leading to a less compact and more transcriptionally active chromatin, and, on the converse, the actions of histone deacetylases (HDAC), which remove the acetyl groups from the lysine residues leading to the formation of a condensed and transcriptionally silenced chromatin. Reversible modification of the terminal tails of core histones constitutes the major epigenetic mechanism for remodeling higher-order chromatin structure and controlling gene expression. HDAC inhibitors (HDI) block this action and can result in hyperacetylation of histones, thereby affecting gene expression. This therapeutic class is able to block angiogenesis and cell cycling, and promote apoptosis and differentiation. HDAC inhibitors both display targeted anticancer activity by themselves and improve the efficacy of existing agents as well as other new targeted therapies.

The HDACs are a family including at least eighteen enzymes, grouped in three classes (Class I, II and III). Class I HDACs include, but are not limited to, HDACs 1, 2, 3, and 8. Class I HDACs can be found in the nucleus and are believed to be involved with transcriptional control repressors. Class II HDACs include, but are not limited to, HDACs 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7.

In some embodiments, the HDAC inhibitor selectively inhibits with a Class I HDAC. As used herein, the term “selective HDAC inhibitor” refers to an HDAC inhibitor that does not significantly interact with all three HDAC classes. As used herein, a “Class I selective HDAC” refers to an HDAC inhibitor that interacts with one or more of HDACs 1, 2, 3 or 8, but does not significantly interact with the Class II HDACs (i.e., HDACs 4, 5, 6, 7 and 9).

In some embodiments, the HDAC inhibitor inhibits a Class I/III HDAC. In some embodiments, the HDAC inhibitor inhibits a Class I/III HDAC.

In some embodiments, the HDAC inhibitor inhibits a Class I or Class III HDAC.

Without limitations, the HDAC inhibitors can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the HDAC inhibitor is a small molecule. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

A number of compounds with HDAC inhibitory activity are known in the art (see e.g., Marks et al., J. Natl. Cancer Inst. 92; 1210-1216 (2000) and Miller et al, J. Med. Chem, 46(24); 5097-5115 (2003), incorporated herein by reference) and can used as an HDAC inhibitory agent of the disclosure. An HDAC inhibitor can be a short-chain fatty acid, such as butyric acid, phenylbutyrate (PB), 4-phenylbutyrate (4-PBA), pivaloyloxymethyl butyrate (Pivanex, AN-9), isovalerate, valerate, valproate, valproic acid, propionate, butyramide, isobutyramide, phenylacetate, 3-bromopropionate, or tributyrin as non-limiting examples. Short-chain fatty acid compounds having HDAC inhibitory activity are described in U.S. Pat. Nos. 4,988,731, 5,212,326, 4,913,906, 6,124,495, 6,110,970 6,419,953, 6,110,955, 6,043,389, 5,939455, 6,511,678, 6,528,090, 6,528,091, 6,713,086, 6,720,004, U.S. Patent Publication No. 20040087652, Intl. Publication No. WO 02/007722, and in Phiel et al, J Biol Chem, 276(39):36734-41 (2001), Rephaeli et al, Int J Cancer, 116(2):226-35 (2005), Reid et al. Lung Cancer, 45(3):381-6 (2004), Gottlicher et al, 2001, EMBO J, 22(13):3411-20 (2003), and Vaisburg et al, Bioorg Med Chem Lett, 14(1):283-7 (2004), content of all of which is incorporated herein by reference in its entirety. An HDAC inhibitor can be compound bearing a hydroxyamic acid group, such as suberoylanlide hydroxamic acid (SAHA), trichostatin A (TSA), trichostatin C (TSC), salicylhydroxamic acid, oxamflatin, suberic bishydroxamic acid (SBHA), m-carboxycinnamic acid bishydroxamic acid (CBHA), pyroxamide (CAS RN 382180-17-8), diethyl bis-(pentamethylene-N,Ndimethylcarboxamide)malonate (EMBA), azelaic bishydroxamic acid (ABHA), azelaic-1-hydroxamate-9-anilide (AAHA), 6-(3-Chlorophenylureido) carpoic hydroxamic acid, or A-161906 as non-limiting examples.

Hydroxyamic acid compounds having HDAC inhibitory activity are described in U.S. Pat. Nos. 6,800,638, 6,784,173, 6,531,472, 6,495,719, 6,512,123, and 6,511,990, U.S. Patent Publication Nos. 20060004041, 20050227976, 20050187261, 20050107348, 20050131018, 20050124679, 20050085507, 20040266818, 20040122079, 20040024067, and 20030018062, Intl. Publication Nos. EP1174438, WO/2004092115, WO/2005019174, WO0052033, W0018045, W0018171, WO0138322, WO0170675, WO9735990, WO9911659, WO0226703, WO0230879 and WO0226696, and in Butler et al, Clin Cancer Res., 7: 962-970 (2001), Richon et al, Proc. Natl. Acad. Sci. USA: 95; 3003-3007 (1998), Kim et al. Oncogene: 18(15); 24612470 (1999), Klan et al, Biol Chem., 384(5):777-85 (2003), Yoshida et al, J Biol Chem., 265(28): 17174-9 (1990), Suzuli et al, Bioorg Med Chem Lett., 15(2):331-5 (2005), Kelly et al, J Clin Oncol., 23(17):3923-31 (2005), Kelly et al, Clin Cancer Res., 9(10 Pt 1):3578-88 (2003), Sonoda et al. Oncogene, 13(1):143-9 (1996), Richon et al, Proc Natl Acad Sci USA., 93(12):5705-8 (1996), Jung et al, J. Med. Chem., 42; 4669-4679. (1999), Jung et al, Bioorg. Med. Chem. Lett, 7(13); 1655-1658 (1997), Lavoie et al, Bioorg. Med. Chem. Letters 11, 2847-2850 (2001), Remiszewski et al, J. Med. Chem. 45, 4, 753-757 (2002), Sternson et al. Org. Lett. 3, 26, 4239-4242 (2001), Bouchain et al, J Med Chem., 46(5):820-30 (2003), and Woo et al, J Med Chem., 45(13):2877-85 (2002).

An HDAC inhibitor can be a cyclic tetrapeptide, such as Depsipeptide (FK228), FR225497, trapoxin A, apicidin, chlamydocin, or HC-toxin as non-limiting examples. Cyclic tetrapeptides having HDAC inhibitory activity are described in U.S. Pat. Nos. 5,922,837, 6,403,555, 6,656,905, 6,399,568, 6,825,317, 6,831,061, U.S. Patent Publication Nos. 20050209134, 20040014647, 20030078369, and 20020120099, and in Kijima et al, J Biol Chem, 268(30):22429-35 (1993), Jose et al, Bioorg Med Chem Ze#,14(21):5343-6 (2004), Xiao et al. Rapid Commun Mass Spectrom., 17(8):757-66 (2003), Furumai et al. Cancer Res., 62(17):4916-21 (2002), Nakajima et al, Exp. Cell Res., 241; 126-133 (1998), Sandor et al, Clin Cancer Res., 8(3):718-28 (2002), Jung et al, J. Med. Chem., 42; 4669-4679. (1999), and Jung et al, Bioorg. Med. Chem. Lett, 7(13); 1655-1658 (1997), content of all of which is incorporated herein by reference in its entirety.

An HDAC inhibitor can be a benzamide, such as MS-275. Benzamides having HDAC inhibitory activity are described in U.S. Pat. Nos. 6,174,905 and 6,638,530, U.S. Patent Publication Nos. 2004005513, 20050171103, 20050131018, and 20040224991, Intl. Publication Nos. WO/2004082638, WO/2005066151, WO/2005065681, EP 0847992 and JP 258863/96, and in Saito et al, Proc. Natl. Acad. Sci. USA, vol. 96, pp. 45924597 (1999); Suzuki et al, J. Med. Chem., vol. 42, pp. 3001-3003 (1999), Ryan et al, J Clin Oncol, 23(17):391222 (2005), Pauer et al. Cancer Invest. 22(6):886-96 (2004), and Undevia et al, Ann Oncol, 15(11): 1705-11 (2004), content of all of which is incorporated herein by reference in its entirety.

An HDAC inhibitor can be a depudecin, a sulfonamide anilide (e.g., diallyl sulfide), BL1521, curcumin (diferuloylmethane), CI-994 (N-acetyldinaline), spiruchostatin A, Scriptaid, carbamazepine (CBZ), or a related compound. These and related compounds having HDac inhibitory activity are described in U.S. Pat. No. 6,544,957, and in Lea et al. Int. J. Oncol, 15, 347-352 (1999), Ouwehand et al, FEBSLett., 579(6):1523-8 (2005), Kraker et al, Mol Cancer Ther. 2(4):401-8 (2003), de Ruijter et al, Biochem Pharmacol, 68(7): 1279-88 (2004), Liu et al. Acta Pharmacol Sin., 26(5):603-9 (2005), Fournel et al. Cancer Res., 62: 4325-4330 (2002), Yurek-George et al, J Am Chem Soc, 126(4):1030-1 (2004), Su et al. Cancer Res., 60(12):3137-42 (2000), Beutler et al. Life Sci., 76(26):3107-15 (2005), and Kwon et al, Proc. Natl. Acad. Sci. USA 95, 3356-3361 (1998), content of all of which is incorporated herein by reference in its entirety.

An HDAC inhibitor can be a compound comprising a cyclic tetrapeptide group and a hydroxamic acid group. Examples of such compounds are described in U.S. Pat. Nos. 6,833,384 and 6,552,065, and in Nishino et al, Bioorg Med Chem., 12(22):5777-84 (2004), Nishino et al. Org Lett, 5(26):5079-82 (2003), Komatsu et al. Cancer Res., 61(11):4459-66 (2001), Furumai et al, Proc Natl Acad Sci USA., 98(1):87-92 (2001), Yoshida et al. Cancer Chemotherapy and Pharmacology, 48 Suppl. 1; S20-S26 (2001), and Remiszeski et al, J Med Chem., 46(21):4609-24 (2003), content of all of which is incorporated herein by reference in its entirety.

An HDAC inhibitor can be a compound comprising a benzamide group and a hydroxamic acid group. Examples of such compounds are described in Ryu et al. Cancer Lett. Jul. 9, 2005 (epub), Plumb et al, Mol Cancer Ther, 2(8):721-8 (2003), Ragno et al, J Med Chem., 47(6):1351-9 (2004), Mai et al, J Med Chem., 47(5):1098109 (2004), Mai et al, J Med Chem., 46(4):512-24 (2003), Mai et al, J Med Chem., 45(9):1778-84 (2002), Massa et al, J Med Chem., 44(13):2069-72 (2001), Mai et al, J Med Chem., 48(9):3344-53 (2005), and Mai et al, J Med Chem., 46(23):4826-9 (2003), content of all of which is incorporated herein by reference in its entirety.

An HDAC inhibitor can be a compound described in U.S. Pat. Nos. 6,897,220, 6,888,027, 5,369,108, 6,541,661, 6,720,445, 6,562,995, 6,777,217, or 6,387,673, 6,693,132, or U.S. Patent Publication Nos. 20060020131, 20060058553, 20060058298, 20060058282, 20060052599, 2006004712, 20060030554, 20060030543, 20050288282, 20050245518, 20050148613, 20050107348, 20050026907, 20040214880, 20040214862, 20040162317, 20040157924, 20040157841, 20040138270, 20040072849, 20040029922, 20040029903, 20040023944, 20030125306, 20030083521, 20020143052, 20020143037, 20050197336, 20050222414, 20050176686, 20050277583, 20050250784, 20050234033, 20050222410, 20050176764, 20050107290, 20040043470, 20050171347, 20050165016, 20050159470, 20050143385, 20050137234, 20050137232, 20050119250, 20050113373, 20050107445, 20050107384, 20050096468, 20050085515, 20050032831, 20050014839, 20040266769, 20040254220, 20040229889, 20040198830, 20040142953, 20040106599, 20040092598, 20040077726, 20040077698, 20040053960, 20040002506, 20030187027, 20020177594, 20020161045, 20020119996, 20020115826, 20020103192, or 20020065282, content of all of which is incorporated herein by reference in its entirety.

Additional non-limiting examples include a reported HDAC inhibitor selected from ONO-2506 or arundic acid (CAS RN 185517-21-9); MGCD0103 (see Gelmon et al. “Phase I trials of the oral histone deacetylase (HDac) inhibitor MGCD0103 given either daily or 3× weekly for 14 days every 3 weeks in patients (pts) with advanced solid tumors. “Journal of Clinical Oncology, 2005 ASCO Annual Meeting Proceedings. 23(16S, June 1 Supplement), 2005: 3147 and Kalita et al. “Pharmacodynamic effect of MGCD0103, an oral isotype-selective histone deacetylase (HDac) inhibitor, on HDac enzyme inhibition and histone acetylation induction in Phase I clinical trials in patients (pts) with advanced solid tumors or non-Hodgkin's lymphoma (NHL)”Journal of Clinical Oncology, 2005 ASCO Annual Meeting Proceedings. 23(16S, Part I of II, June 1 Supplement), 2005: 9631), a reported thiophenyl derivative of benzamide HDac inhibitor as presented at the 97th American Association for Cancer Research (AACR) Annual Meeting in Washington, D.C. in a poster titled “Enhanced Isotype-Selectivity and Antiproliferative Activity of Thiophenyl Derivatives of BenzamideHDac Inhibitors In Human Cancer Cells,” (abstract #4725), and a reported HDac inhibitor as described in U.S. Pat. No. 6,541,661; SAHA or Vorinostat (CAS RN 149647-78-9); PXD101 or PXD 101 or PX 105684 (CAS RN 414864-00-9), CI-994 or Tacedinaline (CAS RN 112522-64-2), MS-275 (CAS RN 209783-80-2), or an inhibitor reported in WO2005/108367, content of all of which is incorporated herein by reference in its entirety.

An HDAC inhibitor can be a novel HDAC inhibitor identified using structure-activity relationships and teachings known in the art and described, e.g., in Miller et al., J. Med. Chem., 46(24); 5097-5115 (2003) and Klan et al., Biol Chem., 384(5):777-85 (2003)), all of which are incorporated herein by reference in their entirety. Methods to assess histone deacetylase activity are known in the art, and are described, e.g., in Richon et al., Methods Enzymol, 376:199-205 (2004), Wegener et al., Mol Genet Metab., 80(1-2): 138-47 (2003), U.S. Pat. No. 6,110,697, and U.S. Patent Publication Nos. 20050118596, 20050227300, 20030161830, 20030224473, 20030082668, 20030013176, and 20040091951), content of all of which is incorporated herein by reference in its entirety.

Antisense oligonucleotides and ribozymes that inhibit transcription and/or translation of one or more HDACs are described in U.S. Pat. No. 6,953,783, and U.S. Patent Publication Nos. 20050171042, 20040266718, 20040204373, 20040077578, 20040077084, 20040077083, 20040072770, 20030236204, 20030216345, 20030152557, 20030148970, 20030078216, 20020137162, 20020164752, 20020115177, and 20020061860, content of all of which is incorporated herein by reference in its entirety.

In some embodiments, the HDAC inhibitor can be selected from the group consisting of valproic acid, MS-275, SAHA, FK228, AN-9, CI-994, LAQ-824, G2M-777, PXD-101, LBH-589, MGCD-0103, MK0683, pyroxamide, sodium phenylbutyrate, CRA-024781, and derivatives, salts, metabolites, prodrugs, and stereoisomers thereof, and any combinations thereof.

In some embodiments, the HDAC inhibitor is valproic acid (inhibits histones 1 and 2a), SAHA also called Verinostat (inhibits histones 1 and 3), MS-275 also called Entinostat (inhibits histones 1, 2, 3, 4, and 8), or a derivative or prodrug thereof. In some embodiments, the HDAC inhibitor can be sodium butyrate, trichostatin A, or a derivative or prodrug thereof.

It is preferred and desirable that a HDAC inhibitor administered with the method described herein does not impair normal wound healing. In other words, it is preferred that a HDAC inhibitor used for treating, inhibiting or reducing adhesion or adhesiogenesis does not act to inhibit or impair normal wound healing mechanisms, e.g., closing of any surgical incision sites.

For administration to a subject, the HDAC inhibitor can be provided in composition, mixture or formulation comprising said HDAC inhibitor. As such, the HDAC inhibitor can be administered to the subject in a soluble form, in a liposome, in a lavage, as a gel or spray, in the form of a physical barrier, or as a controlled-dose formulation.

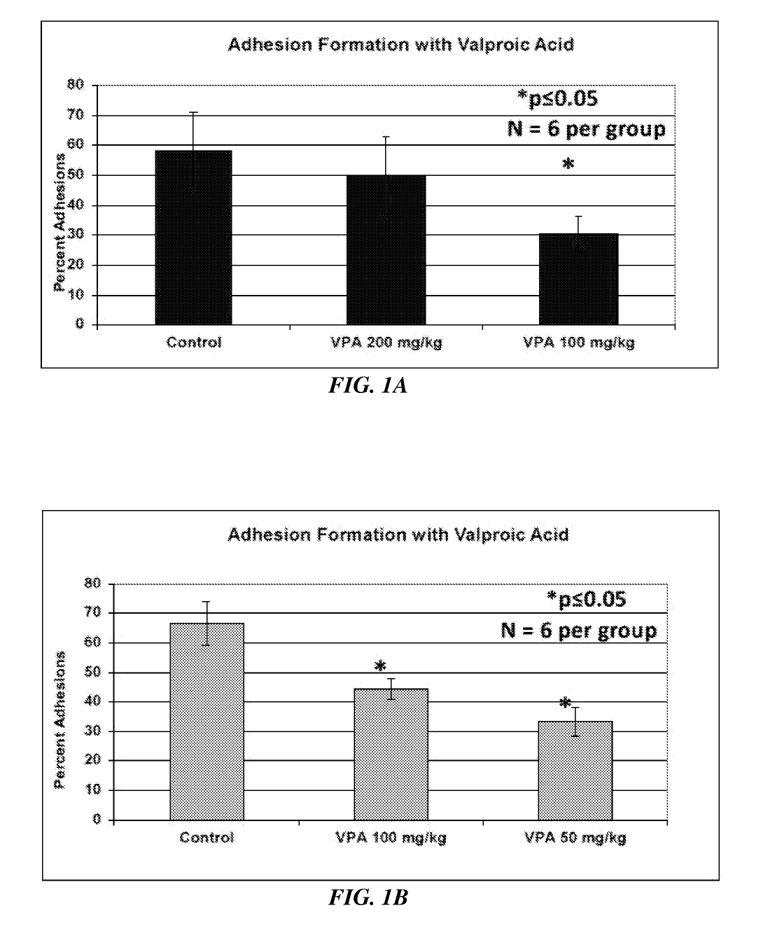

Mixtures and/or formulations for the treatment of subjects can comprise pharmaceutically acceptable carriers and any other compounds or formulations deemed suitable. The specific ratios of the various HDAC inhibitors, other therapeutic agents and/or pharmaceutically acceptable carriers and their dosing can be determined using methods known to the skilled artisan. Some examples of procedures available to determine optimal dosing can be found in the examples below in combination with the discussion of FIGS. 1A-3A. In general, a suitable dose of a particular formulation (and the individual compounds and agents contained therein) will be that amount which is the lowest dose effective to produce a therapeutic effect. However, dosing (of one or more of the compounds and/or agents) can be increased if a beneficial therapeutic effect is observed while toxic effects are avoided or minimized.

For example, for administration to a subject, the HDAC inhibitor can be formulated in pharmaceutically acceptable compositions, e.g., a pharmaceutical compositions, which comprise the HDAC inhibitor formulated together with one or more pharmaceutically acceptable carriers (additives) and/or diluents. As used herein, a “pharmaceutical composition” refers to a chemical or biological composition suitable for administration to a subject (e.g. human or mammal). Such compositions may be specifically formulated for administration via one or more of a number of routes, including but not limited to, oral, parenteral, intravenous, intraarterial, intraperitoneal, subcutaneous, intranasal, sublingual, intraspinal, intracerebroventricular, and the like.

As used here, the phrase “pharmaceutically acceptable” refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The terms “physiologically tolerable carriers” and “biocompatible delivery vehicles” are sometimes used in the literature or herein and are intended to be interchangeable with the phrase “pharmaceutically acceptable carrier”.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Carriers include compounds that facilitate the administration and delivery of another compound and can also facilitate the administration and delivery of another compound and can also facilitate the incorporation of another compound into cells or tissues. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. Dimethyl sulfoxide (DMSO) is a commonly used carrier for improving incorporation of certain organic compounds into cells or tissues.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

Formulations useful in the methods described herein can also include surfactants. Many organized surfactant structures have been studied and used for the formulation of drugs. Suitable surfactants include fatty acids and/or esters or salts thereof, bile acids and/or salts thereof. In certain embodiments, the surfactant can be anionic, cationic, or nonionic. The use of surfactants in drug products, formulations and in emulsions has been reviewed (Rieger, in Pharmaceutical Dosage Forms, Marcel Dekker, Inc., New York, N.Y., 1988, p. 285).

Excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and PRIMOGEL′, and the like.

Formulations for oral administration of the HDAC inhibitors can comprise an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS (N, N-bis(3-D-gluconamidopropyl)-cholamide), chlorobutanol, octoxynol-9, benzyl alcohol, phenols, cresols, and alkyl alcohols. One oral absorption enhancer for the methods described herein is sodium lauryl sulfate. Oral formulations and their preparation are described in detail in U.S. Pat. No. 6,887,906 and No. 6,747,014 and US Patent Application Publication No. 20030027780, content of each of which is incorporated herein by reference in its entirety.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

Suitable formulations also include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, for example in the range of in one embodiment about 0.1 to 10 mg/ml, in another embodiment about 2.0 mg/ml; and/or mannitol or another sugar, for example in the range of in one embodiment 10 to 100 mg/ml, in another embodiment about 30 mg/ml; phosphate-buffered saline (PBS), and any other formulation agents conventional in the art.

In some embodiments, the HDAC inhibitor can be prepared with a carrier that can protect the HDAC inhibitor against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In some embodiments, it can be advantageous to formulate the compositions comprising the HDAC inhibitor in dosage unit form for ease of administration and uniformity of dosage. As used herein, “dosage unit” refers to physically discrete units suited as unitary dosages for the subject to be treated; each unit containing a predetermined quantity of the HDAC inhibitor calculated to produce the desired therapeutic effect in association with the required pharmaceutical carrier.

The formulations can conveniently be presented in unit dosage form and can be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make the formulations include the step of bringing into association or contacting an HDAC inhibitor with one or more excipients or carriers. In general, the formulations are prepared by uniformly and intimately bringing into association one or more compounds with liquid excipients or finely divided solid excipients or both, and then, if appropriate, shaping the product.

As used herein, the term “dosage form” refers to any solid object, semi-solid, or liquid pharmaceutical composition designed to contain a specific pre-determined amount (i.e., dose) of an HDAC inhibitor. Examples of dosage forms include, but are not limited to: solutions; gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

The preparative procedure can include the sterilization of the pharmaceutical preparations. The compounds may be mixed with auxiliary agents such as lubricants, preservatives, stabilizers, salts for influencing osmotic pressure, etc., which do not react deleteriously with the compounds.

Mixtures and/or formulations for the treatment of subjects can comprise pharmaceutically acceptable carriers and any other compounds or formulations deemed suitable. The specific ratios of the various HDAC inhibitors, other therapeutic agents and/or pharmaceutically acceptable carriers and their dosing can be determined using methods known to the skilled artisan. In general, a suitable dose of a particular formulation (and the individual compounds and agents contained therein) will be that amount which is the lowest dose effective to produce a therapeutic effect. However, dosing (of one or more of the compounds and/or agents) can be increased if a beneficial therapeutic effect is observed while toxic effects are avoided or minimized.

The amount of the HDAC inhibitor which can be combined with a carrier material to produce a single dosage form will generally be that amount of the HDAC inhibitor which produces a therapeutic effect. Generally out of one hundred percent, this amount will range from about 0.1% to 99%, preferably from about 5% to about 70%, most preferably from 10% to about 30% (wt/wt) of HDAC inhibitor to total of the composition.

The pharmaceutical composition can be specially formulated for administration in solid, liquid, or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally.

The HDAC inhibitor can be formulated in a gelatin capsule, in tablet form, dragee, syrup, suspension, topical cream, suppository, injectable solution, or kits for the preparation of syrups, suspension, topical cream, suppository or injectable solution just prior to use. Further, an HDAC inhibitor can be included in composites, which facilitate its slow release into the blood stream, e.g., silicon discs, polymer beads, sustained release compositions, and the like.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets can be coated with shellac, sugar, or both. Syrup can contain, in addition to the active ingredient, sucrose as a sweetening agent, methyl and propylparabens as preservatives, a dye, and flavoring such as cherry or orange flavor.

Examples of injectable form include solutions, suspensions and emulsions. Injectable forms also include sterile powders for extemporaneous preparation of injectable solutions, suspensions or emulsions. The compounds of the present invention can be injected in association with a pharmaceutical carrier such as normal saline, physiological saline, bacteriostatic water, CREMOPHOR™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatinA suitable carrier is 5% dextrose in saline. Frequently, it is desirable to include additives in the carrier such as buffers and preservatives or other substances to enhance isotonicity and chemical stability.

In some embodiments, the composition or formulation comprising the HDAC inhibitor is for parenteral administration. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a patient, including, but not limited to, administration of DUROS®-type dosage forms, and dose-dumping.

Suitable vehicles that can be used to provide parenteral dosage forms of the HDAC are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of an HDAC inhibitor can also be incorporated into the parenteral dosage forms of the disclosure, including conventional and controlled-release parenteral dosage forms.

The incorporation of HDAC inhibitors in various sustained release gels and polymers can potentially widen the applicability of the method to laparoscopic procedures as well at cardiac, pulmonary, ocular, otologic and orthopedics procedures where adhesions are also a major problem.

In some embodiments, pharmaceutical compositions can be for use as lavages or gels to coat the peritoneum.

The HDAC inhibitor formulations used according to the methods described herein can include soluble forms, e.g. a lavage for placement in the abdominal cavity prior to completing abdominal surgery. These formulations are herein referred to as “HDAC inhibitor instillate formulations.” These formulations can, for example, be administered intra-abdominally following a surgical procedure into a patient to prevent the formation of post-operative adhesions, or into/onto any other desired wound, disease, etc., site. This liquid can be a solvent and can subsequently produce a solution of the agent. Additionally, the solvent used to dissolve the agent can be water-based. Dissolving the agent in an electrolytic solution can make the instillate formulation. The HDAC inhibitor instillate is then administered to a suitable body cavity where it will prevent the formation of adhesions.

In some embodiments, the HDAC inhibitor instillate solution is a substantially non-viscous liquid, for example having a viscosity substantially similar to water, capable of reaching substantially all areas of a specific body cavity or surgical site where it is introduced. The desired mixture can incorporate at least one agent discussed herein into a liquid to produce a solution (or suspension, etc.) at concentrations of between about 0.0001% w/v and 1% w/v, between 1% w/v and 2% w/v, 2% w/v and 5% w/v, 5% and 10% w/v, 10% w/v and 25% w/v, and 25% w/v and 50% w/v, or other concentrations discussed herein.

Sterile compositions for parenteral direct administration can preferably be aqueous or non-aqueous solutions, suspensions or emulsions. Solvents or vehicles that can be used include water, propylene glycol, a polyethylene glycol, plant oils, in particular olive oil, injectable organic esters, for example ethyl oleate, or other suitable organic solvents. These compositions can also comprise adjuvants, in particular wetting agents, tonicity agents, emulsifiers, dispersants and stabilizers. The sterilization can be performed in several ways, for example by aseptic filtration, by incorporating sterilizing agents into the composition, by irradiation or by heating. They can also be prepared in the form of sterile solid compositions that can be dissolved at the time of use in sterile water or any other injectable sterile medium. The sterile compositions can include sterile aqueous solutions which can also contain buffers, diluents and other suitable additives such as, but not limited to, penetration enhancers, carrier compounds and other pharmaceutically acceptable carriers or excipients. Aqueous suspensions can further contain substances which increase the viscosity of the suspension including, for example, sodium carboxymethylcellulose, sorbitol and/or dextran.

In some embodiments, the HDAC inhibitor can be administrated encapsulated within liposomes. As used herein, a liposome is a structure having lipid-containing membranes enclosing an aqueous interior. Liposomes can have one or more lipid membranes. Liposomes can be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles. Liposomal suspensions (including liposomes targeted to particular cells) can also be used as pharmaceutically acceptable carriers.

Liposomes can be cationic (Wang et al., Biochem. Biophys. Res. Commun., 1987, 147, 980-985), anionic (Zhou et al., Journal of Controlled Release, 1992, 19, 269-274), or nonionic (Hu et al. S.T.P. Pharma. Sci., 1994, 4, 6, 466), content of each of which is incorporated herein by reference in its entirety. Liposomes can comprise a number of different phospholipids, lipids, glycolipids, and/or polymers which can impart specific properties useful in certain applications and which have been described in the art (Allen et al., FEBS Letters, 1987, 223, 42; Wu et al., Cancer Research, 1993, 53, 3765; Papahadjopoulos et al. Ann. N.Y. Acad. Sci., 1987, 507, 64; Gabizon et al. PNAS, 1988, 85, 6949; Klibanov et al. FEBS Lett., 1990, 268, 235; Sunamoto et al. Bull. Chem. Soc. Jpn., 1980, 53, 2778; Illum et al. FEBS Lett., 1984, 167, 79; Blume et al. Biochimica et Biophysica Acta, 1990, 1029, 91; Hughes et al. Methods Mol Biol. 2010; 605:445-59; U.S. Pat. Nos. 4,837,028; 5,543,152; 4,426,330; 4,534,899; 5,013,556; 5,356,633; 5,213,804; 5,225,212; 5,540,935; 5,556,948; 5,264,221; 5,665,710; European Patents EP 0 445 131 B1; EP 0 496 813 B1; and European Patent Publications WO 88/04924; WO 97/13499; WO 90/04384; WO 91/05545; WO 94/20073; WO 96/10391; WO 96/40062; WO 97/0478), content of each of which is incorporated herein by reference in its entirety.

Liposomes can further include one or more additional lipids and/or other components such as cholesterol. Other lipids can be included in the liposome compositions for a variety of purposes, such as to prevent lipid oxidation, to stabilize the bilayer, to reduce aggregation during formation or to attach ligands onto the liposome surface. Any of a number of lipids can be present, including amphipathic, neutral, cationic, and anionic lipids. Further, such lipids can be used alone or in combination.

Additional components that can be present in a liposome can include bilayer stabilizing components such as polyamide oligomers (see, e.g., U.S. Pat. No. 6,320,017), peptides, proteins, detergents, lipid-derivatives, such as PEG conjugated to phosphatidylethanolamine, PEG conjugated to phosphatidic acid, PEG conjugated to ceramides (see, U.S. Pat. No. 5,885,613), PEG conjugated dialkylamines and PEG conjugated 1,2-diacyloxypropan-3-amines.

Liposome can include components selected to reduce aggregation of lipid particles during formation, which can result from steric stabilization of particles which prevents charge-induced aggregation during formation. Suitable components that reduce aggregation include, but are not limited to, polyethylene glycol (PEG)-modified lipids, monosialoganglioside Gm1, and polyamide oligomers (“PAO”) such as (described in U.S. Pat. No. 6,320,017). Exemplary suitable PEG-modified lipids include, but are not limited to, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Particularly preferred are PEG-modified diacylglycerols and dialkylglycerols. Other compounds with uncharged, hydrophilic, steric-barrier moieties, which prevent aggregation during formation, like PEG, Gm1, or ATTA, can also be coupled to lipids to reduce aggregation during formation. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids). It should be noted that aggregation preventing compounds do not necessarily require lipid conjugation to function properly. Free PEG or free ATTA in solution may be sufficient to prevent aggregation. If the liposomes are stable after formulation, the PEG or ATTA can be dialyzed away before administration to a subject.

Neutral lipids, when present in the liposome composition, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in liposomes described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C₁₄ to C₂₂ are preferred. In another group of embodiments, lipids with mono or diunsaturated fatty acids with carbon chain lengths in the range of C₁₄ to C₂₂ are used. Additionally, lipids having mixtures of saturated and unsaturated fatty acid chains can be used. Preferably, the neutral lipids used in the present invention are DOPE, DSPC, POPC, DMPC, DPPC or any related phosphatidylcholine. The neutral lipids useful in the present invention may also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Cationic lipids, when present in the liposome composition, can be any of a number of lipid species which carry a net positive charge at about physiological pH. Such lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N, N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), 5-carboxyspermylglycine diocaoleyamide (“DOGS”), and dipalmitoylphosphatidylethanolamine 5-carboxyspermyl-amide (“DPPES”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other cationic lipids suitable for lipid particle formation are described in WO98/39359, WO96/37194. Other cationic lipids suitable for liposome formation are described, for example in US Patent Application Publication No. 2011/0997720 and PCT Patent Application Publication No. WO 2009/132131 and No. WO 2009/132131, content of all of which is incorporated herein by reference in its entirety.

Anionic lipids, when present in the liposome composition, can be any of a number of lipid species which carry a net negative charge at about physiological pH. Such lipids include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

As used herein, the term “Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Also suitable for inclusion in the liposome compositions described herein are programmable fusion lipids. Liposomes containing programmable fusion lipids have little tendency to fuse with cell membranes and deliver their payload until a given signal event occurs. This allows the liposome to distribute more evenly after administration into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the liposome membrane over time. By the time the liposome is suitably distributed in the body, it has lost sufficient cloaking agent so as to be fusogenic. With other signal events, it is desirable to choose a signal that is associated with the disease site or target cell, such as increased temperature at a site of inflammation.

A liposome can also include a targeting moiety, e.g., a targeting moiety that is specific to a cell type or tissue. Targeting of liposomes with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). Other targeting moieties, such as ligands, cell surface receptors, glycoproteins, vitamins (e.g., riboflavin), aptamers and monoclonal antibodies, can also be used. The targeting moieties can include the entire protein or fragments thereof. Targeting mechanisms generally require that the targeting agents be positioned on the surface of the liposome in such a manner that the targeting moiety is available for interaction with the target, for example, a cell surface receptor.

In one approach, a targeting moiety, such as receptor binding ligand, for targeting the liposome can be linked to the lipids forming the liposome. In another approach, the targeting moiety can be attached to the distal ends of the PEG chains forming the hydrophilic polymer coating (Klibanov, et al., Journal of Liposome Research 2: 321-334 (1992); Kirpotin et al., FEBS Letters 388: 115-118 (1996)). A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002). Other lipids conjugated with targeting moieties are described in US provisional application #61/127,751 (filed May 14, 2008) and PCT application #PCT/US2007/080331 (filed Oct. 3, 2007), content of all of which is incorporated by reference in its entirety.

A liposome composition can be prepared by a variety of methods that are known in the art. The manufacture of liposomes and insertion of molecules into such liposomes being well known in the art, for example, as described in U.S. Pat. No. 4,522,811, content of which is incorporated herein by reference in its entirety. Additional references pertaining to preparing liposomal formulations include, but are not limited to, U.S. Pat. No. 4,235,871, No. 4,897,355 and No. 5,171,678; published PCT applications WO 96/14057 and WO 96/37194; Felgner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757, content of all of which is incorporated herein by reference in its entirety.

For example, a liposome composition can be prepared by first dissolving the lipid components of a liposome in a detergent so that micelles are formed with the lipid component. The detergent can have a high critical micelle concentration and maybe nonionic. Exemplary detergents include, but are not limited to, cholate, CHAPS, octylglucoside, deoxycholate and lauroyl sarcosine. The HDAC inhibitor preparation e.g., an emulsion, is then added to the micelles that include the lipid components. After condensation, the detergent is removed, e.g., by dialysis, to yield a liposome containing the HDAC inhibitor.

In another example, liposomes can be prepared by diffusing a lipid derivatized with a hydrophilic polymer into preformed liposome, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the HDAC inhibitor is first dispersed by sonication in a lysophosphatidylcholine or other low CMC surfactant (including polymer grafted lipids). The resulting micellar suspension of HDAC inhibitor is then used to rehydrate a dried lipid sample that contains a suitable mole percent of polymer-grafted lipid or cholesterol. The lipid and active agent suspension is then formed into liposomes using extrusion techniques as are known in the art. The resulting liposomes can then be separated from the unencapsulated solution by standard column separation.

The liposomes can be prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323, content of which is incorporated herein by reference in its entirety.

The compositions for use with the methods described herein can be prepared and formulated as emulsions or microemulsions. Emulsions are typically heterogeneous systems of one liquid dispersed in another in the form of droplets usually exceeding 0.1 μm in diameter and have been described in the art. Microemulsion can be defined as a system of water, oil and amphiphile which is a single optically isotropic and thermodynamically stable liquid solution and can comprise surfactants and cosurfactants. Both of these drug delivery means have been described in the art (see e.g., Ansel's Pharmaceutical Dosage Forms and Drug Delivery Systems, Allen, L V., Popovich N G., and Ansel HC., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 199, 245, & 335; Higuchi et al., in Remington's Pharmaceutical Sciences, Mack Publishing Co., Easton, Pa., 1985, p. 301; Leung and Shah, in: Controlled Release of Drugs: Polymers and Aggregate Systems, Rosoff, M., Ed., 1989, VCH Publishers, New York, pages 185-215; Schott, in Remington's Pharmaceutical Sciences, Mack Publishing Co., Easton, Pa., 1985, p. 271; Constantinides et al., Pharmaceutical Research, 1994, 11, 1385-1390; Ritschel, Meth. Find. Exp. Clin. Pharmacol., 1993, 13, 205; Ho et al., J. Pharm. Sci., 1996, 85, 138-143; Lee et al., Critical Reviews in Therapeutic Drug Carrier Systems, 1991, p. 92; U.S. Pat. Nos. 6,191,105; 7,063,860; 7,070,802; 7,157,099).

The HDAC inhibitor composition and formulations used according to the methods described herein can also include compositions or formulations that will coat or spray a tissue surface(s). Non-limiting examples of such formulations include sprays, films, gels, hydrogels, creams, pastes, and ointments. Such formulations can comprise a controlled-dosage form of the HADC inhibitor, e.g., a biodegradable hydrogel comprising an HDAC inhibitor.

As used herein, sprays or aerosols refer to solutions, suspensions or emulsions of an HDAC inhibitor as described herein in a pharmaceutically acceptable solvent such as, in particular, ethanol or water, or a mixture of such solvents that can be sprayed onto a site of adhesion, such as an organ surface. The formulation can, if required, also contain other pharmaceutical excipients such as surfactants, emulsifiers and stabilizers, and a propellant gas. Such a preparation contains, for example, the active ingredient in a concentration of about 0.1 to 10, in particular of about 0.3 to 3% by weight. In some embodiments, a spray formulation comprising the HDAC inhibitor that turns into a film or coating upon contact with a surface, e.g., organ or tissue surface, can be used to coat organ surfaces or to line the tissues of a surgical site for use with the methods described herein.

The HDAC inhibitor can be formulated as a film suitable for direct application to tissue of a subject for the treatment of adhesions. The desired properties of the film include that it is thin, flexible, has the ability to be handled and is able to be affixed to tissue. The HDAC inhibitor can also be incorporated into a polymer to create a film. The properties of the polymeric film formulation can be enhanced with the addition of suitable excipients. In some embodiments, and HDAC inhibitor can be combined with hyaluronic acid polymer to make a film. Excipients which can be added include 1-ethyl-3-[3-(dimethylamino)propyl]carbodiimide (EDAC) and glycerol. One embodiment of the methods descried herein comprises the incorporation of the HDAC inhibitor to produce a 0.001%-99% w/w drug (agent) loaded film.

Gel formulations of the HDAC inhibitor refer to semisolid, suspension-type systems. Single-phase gels contain organic macromolecules distributed substantially uniformly throughout the carrier liquid, which is typically aqueous, but also can contain an alcohol and, optionally, an oil. A gel can be administered to a body cavity of a subject. Desired properties of the gel include that it is viscous enough to be applied to a specific location and remain affixed there, thus it will not flow under its own weight; and that it can be administered to the preferred location with the use of a syringe or injected through a needle. The HDAC inhibitor can be incorporated to yield a 0.001%-1% w/v gel. The HDAC inhibitor can also be integrated to produce a 1%-10% w/v gel. The HDAC inhibitor can also be loaded to produce a 10%-50% w/v gel, or other concentrations discussed herein.

The methods of the invention described herein can also comprise administering a hydrogel comprising an HDAC inhibitor. Commercially available hydrogels can be supplied either as a dry powder or a partially hydrated paste intended for administration after dispersion in an appropriate amount of aqueous vehicle. These powders are formed by mechanical disruption of cross-linked matrices, such as absorbable gelatin sponges, U.S.P. (e.g., Gelfoam®, Pfizer, Inc. or Surgifoam™, Ethicon, Inc.), or the cakes that are formed during typical chemical or dehydrothermal cross-linking treatment (see, e.g., U.S. Pat. No. 6,063,061; U.S. Patent application pub. No. 2003/0064109). These hydrogels can be based on gelatin, collagen, dextran, chitosan. Other compositions are also used, for example, alginate (U.S. Pat. No. 5,294,446) and synthetic polymers such as polyphosphazines, polyacrylates, polyanhydrides, and polyorthoesters, as well as “block copolymers” such as mixtures of polyethylene oxide and polypropylene glycol (U.S. Pat. Nos. 5,041,138; 5,709,854; 5,736,372). In addition, U.S. Pat. Nos. 5,749,874 and 5,769,899 (both Schwartz et al, 1998) disclose two-component implants, where one component is an anchoring device, made of a relatively hard yet biodegradable material (such as polyglycolic acid, polylactic acid, or combinations thereof), which helps secure and anchor the hydrogel implants and a second component that comprises a more porous and flexible matrix.

Hydrogels can be administered dry, partially hydrated, or fully hydrated. In the fully hydrated state, the hydrogel cannot absorb further fluid, and is fully swollen in size. In contrast, a dry or partially hydrated hydrogel composition has excess adsorptive capacity. Upon administration, dry or partially hydrated hydrogel will absorb fluid leading to a swelling of the gelatin matrix in vivo. Swelling of dry or partially hydrated hydrogel should be considered in the context of administration.

Ointments refer to semisolid preparations that are typically based on petrolatum or other petroleum derivatives. The specific ointment base to be used, as will be appreciated by those skilled in the art, is one that will provide for optimum drug delivery, and, preferably, can provide for other desired characteristics as well, e.g., emolliency. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (OW) emulsions, and include, for example, cetyl alcohol, glyceryl monostearate, lanolin and stearic acid. Preferred water-soluble ointment bases are prepared from polyethylene glycols of varying molecular weight (Remington: The Science and Practice of Pharmacy).

Creams containing the selected ARB agent are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra, is generally a nonionic, anionic, cationic or amphoteric surfactant.

In some embodiments, the HDAC inhibitor can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the HDAC inhibitor for use in the methods described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

Any suitable administration can be used for administering the HDAC inhibitor to the subject. As used herein, the term “administer” refers to the placement of an agent, e.g., a HDAC inhibitor, or a composition comprising the agent into a subject by a method or route which results in at least partial localization of the agent at a desired site. Without limitations, the in vivo administration can be by any appropriate route which results in some localization of the agent at the desired site in the subject, i.e., administration results in delivery to a desired location in the subject where at least some amount or portion of the agent is delivered. Routes of administration can be accomplished through any means known by those skilled in the art. Such means include, but are not limited to, oral, buccal, intravenous, subcutaneous, intramuscular, direct administration, and the like. Exemplary modes of administration include, but are not limited to, implant, injection, infusion, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion.

As used herein, the terms “administered” and “subjected” are interchangeable with respect to the treatment of a disease or disorder and both terms refer to a subject being treated with an effective dose of pharmaceutical composition comprising a HDAC inhibitor by methods of administration such as parenteral or systemic administration.

Suitable forms of administration for use with the methods described herein include, but are not limited to, peritoneal, subcutaneous or oral administration.

The administration can be local or systemic. The phrases “systemic administration,” “administered systemically”, “peripheral administration” and “administered peripherally” as used herein refer to the administration of the agent other than directly into a target site, tissue, or organ, such as a surgical site, such that it enters the subject's circulatory system and, thus, is subject to metabolism and other like.

In some embodiments, administration can be local. By local administration is meant the administration is local administered directly to the site of an adhesion and refers to administration of an HDAC inhibitor into or near a tissue at risk for or having an adhesion. Without wishing to be bound by a theory, local administration provides a high local (regional) concentration of the HDAC inhibitor at or near the site of adhesion. Such local administration can be accomplished via injection (i.e., parenteral) or by placing a composition or formulation comprising the HDAC inhibitor at the expected site of adhesion or adhesiogenesis, e.g. treating the surface of the peritoneum prior to completing an abdominal surgery, or during or following the abdominal surgery. Accordingly, in some embodiments, the HDAC inhibitor is administered directly to a site of an expected adhesion (for example, a surgical site or tissue) or an adhesion. The local administration can be in the form of topical administration at a site of injury or surgery.

In some embodiments, administration can be parenteral administration. The phrases “parenteral administration” and “administered parenterally” as used herein, refer to modes of administration other than enteral and topical administration, usually by injection, and includes, without limitation, intravenous, intraperitoneal, intramuscular, intraarterial, intrathecal, intraventricular, intracapsular, intraorbital, intracardiac, intradermal, transtracheal, subcutaneous, subcuticular, intraarticular, subcapsular, subarachnoid, intraspinal, intracerebro spinal, and infrasternal injection and infusion.

For parenteral administration, solutions or suspensions of the HDAC inhibitors can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

In some embodiments, administration can be oral administration. The HDAC inhibitors can be orally administered, for example, with an inert diluent, or with an assimilable edible carrier, or they can be enclosed in hard or soft shell capsules, or they can be compressed into tablets, or they can be incorporated directly with the food of the diet. For oral therapeutic administration, the HDAC inhibitors can be incorporated with excipients and used in the form of tablets, capsules, elixirs, suspensions, syrups, and the like. Such compositions and preparations should contain at least 0.1% of compound. The percentage of the HDAC inhibitor in these compositions can, of course, be varied and can conveniently be between about 2% to about 60% of the weight of the unit. The amount of the HDAC inhibitor in such therapeutically useful compositions is such that a suitable dosage will be obtained. Preferred compositions according to the present invention are prepared so that an oral dosage unit contains between about 100 and 2000 mg of the HDAC inhibitor.

In some embodiments, administration can be in the form of a bolus dose, to maximize the circulating levels for the greatest length of time after the dose. Continuous infusion can also be used after the bolus dose.

In some embodiments, the HDAC inhibitor can be implanted into the patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is incorporated herein by reference in its entirety.

The HDAC inhibitor can be administered before or prior to, during, or following another medical treatment (e.g., surgery or surgical implantation), or can be administered following an injury or trauma. Further, timing of HDAC inhibitor administration can be selected, without limitations, based upon factors such as desired routes of administration, dosages desired, type of surgery, whether the surgery was elective or due to an emergency, extent of the surgical procedure, the subject's responsiveness to treatment and other parameters that are assessed by one of ordinary skill in the art in selecting a course of treatment for a particular subject.

In some embodiments, the HDAC inhibitor is applied to the tissues and/or organs to be affected by a surgical procedure in order to reduce or minimize the incidence and/or occurrence of adhesions during and/or following the surgery. In some embodiments, the HDAC inhibitor is administered to the subject during surgery. In some embodiments, the HDAC inhibitor is administered after surgery. In some embodiments, the HDAC inhibitor is administered directly to a body cavity affected by the surgical procedure or a surgical site following a surgery. In some embodiments, the HDAC inhibitor is applied to an incision(s) created during a surgical procedure.

In some embodiments, the HDAC inhibitor is administered to the subject during or following a surgical procedure. In some such embodiments, the surgical procedure comprises introducing an implant into the subject.

In some such embodiments, the HDAC inhibitor is administered to the subject one or more times during the period of 1 hour-10 days prior to the surgical procedure.

In some such embodiments, the HDAC inhibitor is administered to the subject one or more times during the surgical procedure.

In some such embodiments, the HDAC inhibitor is administered to the subject one or more times during the period of 1 hour-10 days following the surgical procedure.

In some embodiments, the methods for the treatment of adhesions as described herein can also be used in combination with any other therapy known in the art for the reduction of adhesion formation or conditions which can cause adhesiogenesis. Accordingly, the HDAC inhibitor can be administrated to a subject in combination with one or more additional pharmaceutically active agents (e.g., therapeutic agents), i.e. the HDAC inhibitor can be administered as the primary therapeutic agent or can be co-administered with one or more additional therapeutic agents. Exemplary pharmaceutically active compound include, but are not limited to, those found in Harrison's Principles of Internal Medicine, 13^{th} Edition, Eds. T.R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

The HDAC inhibitor and the additional agent can be administrated to the subject in the same pharmaceutical composition or in different pharmaceutical compositions. Further, the HDAC inhibitor and the additional agent can be administered (at the same time or at different times.

In some embodiments, the additional therapeutic agent can be an agent useful in treating adhesions or an agent useful in treating a condition which can cause adhesion or adhesiogenesis (e.g. surgery, trauma, or fibrotic disorder) or a symptom or complication of a condition which can cause adhesion or adhesiogenesis. Examples of compounds useful in reducing, inhibiting, minimizing, treating, or preventing adhesion formation include, but are not limited to, sunitinib (sunitinib malate; SUTENT®); Axitinib (AG013736); Sorafenib (BAY 43-9006); ZD1839 (Beringer; genfitinib; IRESSA®); Erlotinib (OSI-774; TARCEVA®); Semaxanib (SU 5416). These compounds are described in U.S. Pat. Nos. 6,573,293; 6,531,491; 7,235,576; 5,457,105; 5,747,498; 5,792,783, content of each of which is incorporated herein by reference in its entirety. Further examples of compounds useful in treating adhesions include, but are not limited to those compositions described in U.S. Pat. Nos. 7,645,733; 5,601,579; RE37828; 5,679,658; 5,478,837; 5,498,613; 5,534,261; 5,614,515; 5,639.468 and US Patent Publication Nos: 2010/0163781; 2006/0251702; 2003/0153529; 2010/0260819; 2011/0076324; 2004/0037866; 2009/0011978; 2006/0269573; each of which is incorporated herein by reference in its entirety.

Accordingly, in some embodiments, the method further comprises administering, along with or concurrently with the HDAC inhibitor, one or more additional agents used to minimize adhesions or ameliorate the condition causing the adhesions. By way of example, in the case of a subject having a trauma or injury involving incisions or a compromised epidermis, the subject can be administered the HDAC inhibitor along with one or more prophylactic or therapeutic antibiotics.

The use of physical barriers to treat adhesions is well known to those of skill in the art. The concurrent use of physical barriers and the HDAC inhibitor compositions and formulations described herein is specifically contemplated for some embodiments of the methods described herein. An HDAC inhibitor can be administered by any of the means described herein in addition to the placement of physical barriers. Furthermore, the physical barriers themselves can be coated and/or impregnated with any of the HDAC inhibitor compositions or formulations described herein.

Physical barriers for the treatment of adhesions can comprise a variety of materials, including, but not limited to carboxymethlycellulose, chitosan, alginate, hyaluronic acid, lipid emulsions, colloidal osmotic agents such as maltodextrindextrose and oxidized, regenerated cellulose. Such physical barriers, their manufacture and their use are described in U.S. Pat. Nos. 5,795,584; 6,500,777; 5,593,441; 6,150,581; 5,580,923; 4,840,626; 6,391,939; 6,693,089; 5,711,958; 6,613,325; 5,007,916; and US Patent Publication Nos: 2011/0052712; 2008/0086216; 2008/0109017; 2010/0183697; 2006/0067976; 2008/0300319, content of all of which is incorporated herein by reference in its entirety.

The methods described herein comprise administering a therapeutically effective amount of an HDAC inhibitor to a subject in need of treatment of adhesion formation or reformation. As used herein, a “therapeutically effective amount” is an amount of a composition comprising an HDAC inhibitor sufficient to produce a measurable anti-adhesion response. Actual dosage levels of active ingredients in a therapeutic composition for use with the methods described herein can be varied so as to administer an amount of the active HDAC inhibitor that is effective to achieve the desired therapeutic response for a particular subject. Generally, the dosage can vary with the age, condition, and sex of the patient and can be determined by one of ordinary skill in the art. Determination and adjustment of a therapeutically effective dose, as well as evaluation of when and how to make such adjustments, are known to those of ordinary skill in the art.

Further, the selected dosage level will depend upon a variety of factors including the activity of the therapeutic composition, formulation, the route of administration, combination with other drugs or treatments, expected adhesion size and the physical condition and prior medical history of the subject being treated. For example, the dosage ranges for the administration of an HDAC inhibitor depend upon the form of the HDAC inhibitor and its potency, as described further herein, and are amounts large enough to produce the desired effect in which the symptoms, markers, signs, and/or incidence of adhesions are reduced. Preferably, the dosage should not be so large as to cause substantial adverse side effects.

Guidance regarding the efficacy and dosage which will deliver a therapeutically effective amount of a HDAC inhibitor to treat adhesions can be obtained from animal models of adhesions. For example, effective doses can be extrapolated from dose-response curves derived from, for example, animal model test bioassays or systems.

The therapeutically effective dose can be estimated initially from cell culture assays. A dose can be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, and immunological assays.

Toxicity and therapeutic efficacy of the HDAC inhibitor can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

Therapeutic compositions comprising an HDAC inhibitor can be tested in one or more appropriate in vitro and/or in vivo animal models of disease, such as the model of surgical adhesions described herein, to confirm efficacy, evaluate tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD₅₀ of the relevant formulation, and/or observation of any side-effects of HDAC inhibitor at various concentrations, e.g., as applied to the mass and overall health of the patient. In determining the effective amount of an HDAC inhibitor to be administered in the treatment of adhesions, the physician evaluates, among other criteria, circulating plasma levels, formulation toxicities, and progression of the condition.

Dosages for a particular patient or subject can be determined by one of ordinary skill in the art using conventional considerations, (e.g. by means of an appropriate, conventional pharmacological protocol). A physician can, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum or tissue half-life of the HDAC inhibitor, or functional derivatives thereof, and the condition of the patient, as well as, for example, the body weight of the patient to be treated. The size of the dose is also determined by the existence, nature, and extent of any adverse side-effects that accompany the administration of a particular composition, formulation, or the like in a particular subject.

The dosage of an HDAC inhibitor administered according to the method described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to the treatment regimen.

Further, for any particular subject, specific dosage regimes can be adjusted over time according to the individual need and the professional judgment of the person administering or supervising the administration of the compositions. For example, the dosage of the therapeutic can be increased if the lower dose does not provide sufficient therapeutic activity.

Generally, the compositions are administered so that HDAC inhibitor is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. In some embodiments, the dose range is from 1 mg/kg to 500 mg/kg, from 10 mg/kg to 400 mg/kg, from 20 mg/kg to 300 mg/kg, or from 30 mg/kg to 200 mg/kg body weight.

In some embodiments of the methods described herein, a minimally therapeutic dose of an HDAC inhibitor is administered. The term “minimally therapeutic dose” refers to the smallest dose, or smallest range of doses, determined to be a therapeutically effective amount as that term is used herein.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the HDAC inhibitor. The HDAC inhibitor can be administered in a single dose or subdivided into smaller sub-doses. Further, the desired dose can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or more times daily.

Further, the HDAC inhibitor can be administered over a period of time, such as over a 5 minute, 10 minute, 15 minute, 20 minute, or 25 minute period. The administration can be repeated, for example, on a regular basis, such as hourly for 3 hours, 6 hours, 12 hours or longer or such as biweekly (i.e., every two weeks) for one month, two months, three months, four months or longer. When multiple doses are administered, the doses can be separated from one another by, for example, one hour, three hours, six hours, eight hours, one day, two days, one week, two weeks, or one month. After an initial treatment regimen, the treatments can be administered on a less frequent basis. For example, following a surgical procedure, after administration biweekly for three months, administration can be repeated once per month, for six months or a year or longer. In some embodiments, administration is chronic, e.g., one or more doses daily over a period of weeks or months.

In some embodiments, the dosing regimen can be intraoperatively (at the conclusion of either a laparotomy or laparoscopic procedure) or postoperatively.

As used herein, a “subject” or “individual” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments, the subject or the patient is human. In some embodiments, the subject does not include a human.

In some embodiments, the subject can be a patient or other subject in a clinical setting.

In some embodiments, a subject in need of a treatment to reduce the incidence or severity of adhesions is a subject who has undergone, undergoing, or is anticipated to undergo a surgery or surgical procedure, such as a surgery for implanting a medical device. The surgery can be of any type known to result in post-operative adhesion.

In some embodiments, a subject in need of a treatment to reduce the incidence or severity of adhesions is a subject who has undergone, undergoing, or is anticipated to undergo a surgery or surgical procedure as a result of a traumatic injury.

In some embodiments of this aspect and all such aspects described herein, the subject has a fibrotic disorder.

In some embodiments, the method further comprise first diagnosing the subject, such as a human subject, as having a fibrotic disease, or having adhesion formation as a result of surgery, or having experienced a trauma that can cause the formation of adhesions.

Subjects having experienced a trauma can be identified or diagnosed by a physician using known methods of diagnosing trauma. Symptoms and/or complications of trauma which characterize these conditions and aid in diagnosis include, but are not limited to, the presence of incisions, wounds, bruises, hematomas, burns, broken bones, and evidence of crush injuries.

Subjects having a fibrotic disease can be identified or diagnosed by a physician using known methods of diagnosing a fibrotic disease. Symptoms and/or complications of fibrotic diseases which characterize these conditions and aid in diagnosis are known to those of ordinary skill in the art. By way of example, in the case of pulmonary fibrosis, symptoms include, but are not limited to, shortness of breath, coughing, fatigue, discomfort and weight loss. Tests that can aid in a diagnosis of pulmonary fibrosis include, but are not limited to, lung biopsy, a videoscopic assisted thoracoscopic wedge biopsy (VATS), and spirometry.

As used herein, the term “normal healthy subject” refers to a subject who has no symptoms of any diseases or disorders, or who is not identified with any diseases or disorders, or who is not on any medication treatment, or a subject who is identified as healthy by physicians based on medical examinations.

With respect to the therapeutic methods described herein, it is not intended that the administration of the HDAC inhibitor be limited to a particular mode of administration, dosage, or frequency of dosing; all modes of administration, including intramuscular, intravenous, inhalation, intranasal, oral, intraperitoneal, intravesicular, intraarticular, intralesional, subcutaneous, or any other route sufficient to provide a dose adequate to treat adhesions are contemplated for use with the methods described herein.

Without wishing to be bound by a theory, the methods, compounds, and compositions described herein can provide widespread adhesion reduction efficacy throughout the peritoneal cavity by a single intraoperative dose. This can provide advantageous results over currently approved physical barriers which are only effective where they are placed and have no remote adhesion reducing effects throughout the peritoneum. In addition, since the HDAC inhibitors are soluble, they can be used in other applications, such as laparoscopic abdominal surgery as well as other surgical procedures such as pulmonary, cardiovascular, orthopedic and ob-gyn.

It should be further understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

The invention can be illustrated by the following numbered paragraphs:
1. A method of treating or inhibiting adhesion formation or reformation in a subject, the method comprising administering to a subject in need thereof a therapeutically effective amount of an histone deacetylase (HDAC) inhibitor.
2. The method of paragraph 1, wherein the HDAC inhibitor is administered directly into the peritoneal cavity to a site of the adhesion.
3. The method of any of paragraphs 1 or 2, wherein said administration is in a single intraoperative dose.
4. The method of paragraphs 1-3, wherein the HDAC inhibitor is administered in a soluble form.
5. The method of any of paragraphs 1-3, wherein the HDAC inhibitor is administered in a controlled-dose or sustained release formulation.
6. The method of any of paragraphs 1-3, wherein the HDAC inhibitor is administered as a gel or a spray.
7. The method of any of paragraphs 1-3, wherein the HDAC inhibitor is administered in a liposome formulation.
8. The method of any of paragraphs 1-3, wherein the HDAC inhibitor is administered as a physical barrier.
9. The method of any of paragraphs 1-3, wherein the HDAC inhibitor is applied to a biodegradable barrier that is placed in the subject during surgery.
10. The method of any of paragraphs 1-3, wherein the HDAC inhibitor is administered as a lavage.
11. The method of any of paragraphs 1-10, wherein the HDAC inhibitor is administered topically at the site of an injury or surgery.
12. The method of any of paragraphs 1-11, wherein the HDAC inhibitor is formulated into a composition which further comprises a pharmaceutically acceptable carrier.
13. The method of any of paragraphs 1-12, wherein the subject has undergone or is undergoing surgery.
14. The method of paragraph 13, wherein the subject has undergone or is undergoing surgery as a result of a traumatic injury.
15. The method of paragraph 14, wherein the traumatic injury is not caused by a surgical procedure.
16. The method of any of paragraphs 1-15, wherein the subject has a fibrotic disorder.
17. The method of any of paragraphs 1-16, wherein the adhesion is a surgical adhesion.
18. The method of any of paragraphs 1-17, wherein the adhesion is an intra-abdominal or peritoneal adhesion.
19. The method of any of paragraphs 1-18, wherein the subject is a mammal.
20. The method of paragraph 19, wherein the subject is a human.
21. The method of any of paragraphs 1-20, wherein the HDAC inhibitor is selected from the group consisting of small organic or inorganic molecules, saccharines, oligosaccharides, polysaccharides, peptides, proteins, peptide analogs and derivatives, peptidomimetics, antibodies, functional portions of antibodies, nucleic acids, nucleic acid analogs and derivatives, an extract made from biological materials, naturally occurring or synthetic compositions, and any combinations thereof.
22. The method of any of paragraphs 1-21, wherein the HDAC inhibitor is Valproic acid (VPA), SAHA, MS-275, or any combinations thereof.
23. The method of any of paragraphs 1-22, further comprising administering a therapeutic agent to the subject.
24. The method of any of paragraphs 1-23, wherein the HDAC inhibitor is administered to the subject during or following a surgical procedure.
25. The method of paragraph 24, wherein the surgical procedure comprises introducing an implant into the subject.
26. The method of paragraphs 24 or 25, wherein the HDAC inhibitor is administered to the subject one or more times during the period of 1 hour-10 days prior to the surgical procedure.
27. The method of any of paragraphs 24-26, wherein the HDAC inhibitor is administered to the subject one or more times during the surgical procedure.
28. The method of any of paragraphs 24-27, wherein the HDAC inhibitor is administered to the subject one or more times during the period of 1 hour-10 days following the surgical procedure.
29. Use of a HDAC inhibitor for the manufacture of a medicament for treatment or reduction of surgical adhesions.
30. The use of paragraph 29, wherein the medicament further comprises at least one therapeutic agent.
31. A method comprising contacting a subject with an HDAC inhibitor during surgery, wherein said contacting is performed by way of an intraperitoneal injection, thereby treating adhesion in the subject.

### SOME SELECTED DEFINITIONS

For convenience, certain terms employed herein, in the specification, examples and appended claims are collected herein below. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein, an “agent” is a chemical molecule of synthetic or biological origin. For example, an agent can be a HDAC inhibitor. In the context of the present invention, an agent can be a molecule that can be used in a pharmaceutical composition. In some embodiments, the agent can be used solely to implement the invention, e.g. by formulating the HDAC inhibitors into compositions that may (or may not) further comprise a pharmaceutically acceptable carrier.

As used herein, the term “agent” refers to any entity which is normally not present or not present at the levels being administered to a cell, tissue or subject. Agent can be a chemical molecule of synthetic or biological origin. Agent can be selected from a group comprising: chemicals; small molecules; nucleic acid sequences; nucleic acid analogues; proteins; peptides; aptamers; antibodies; or functional fragments thereof. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds. In some embodiments, the agent can be a HDAC inhibitor. In the context of the method described herein, an agent can be a molecule that can be used in a pharmaceutical composition. In some embodiments, the agent can be used solely to implement the method described herein, e.g. by formulating the HDAC inhibitors into compositions that may (or may not) further comprise a pharmaceutically acceptable carrier.

As used herein, “efficacy” of a therapeutic agent refers to the relationship between a minimum effective dose and an extent of toxic side effects. Efficacy of an agent is increased if a therapeutic end point can be achieved by administration of a lower dose or a shorter dosage regimen. If toxicity can be decreased, a therapeutic agent can be administered on a longer dosage regimen or even chronically with greater patient compliance and improved quality of life. Further, decreased toxicity of an agent enables the practitioner to increase the dosage to achieve the therapeutic endpoint sooner, or to achieve a higher therapeutic endpoint.

As used herein the term “effective amount” refers to the amount of at least one therapeutic agent (e.g. small molecule compound) or pharmaceutical composition (e.g. a formulation) to reduce or stop at least one symptom of the abnormal proliferation, for example an adhesion. For example, an effective amount using the methods as disclosed herein may be considered as the amount sufficient to reduce a symptom of an adhesion by at least 10%. An effective amount as used herein may also include an amount sufficient to prevent or delay the development of an adhesion. Accordingly, the term “effective amount” or “therapeutically effective amount” refers to the amount of therapeutic agent to reduce or alleviate adhesions. The dosage administered, as single or multiple doses, to a subject will vary depending upon a variety of factors, including pharmacokinetic properties of a small molecule compound, the route of administration, conditions and characteristics (sex, age, body weight, health, size) of subjects, extent of symptoms, concurrent treatments, frequency of treatment and the effect desired.

As used herein, the term “derivative” refers to a chemical substance related structurally to another, i.e., an “original” substance, which can be referred to as a “parent” compound. A “derivative” can be made from the structurally-related parent compound in one or more steps. The general physical and chemical properties of a derivative are also similar to the parent compound.

In some embodiments, the HDAC inhibitor can be in the form of a prodrug. The term “prodrug” as used herein refers to compounds that can be converted via some chemical or physiological process (e.g., enzymatic processes and metabolic hydrolysis) to compound described herein. Thus, the term “prodrug” also refers to a precursor of a biologically active compound that is pharmaceutically acceptable. A prodrug can be inactive when administered to a subject, i.e. an ester, but is converted in vivo to an active compound, for example, by hydrolysis to the free carboxylic acid or free hydroxyl. A prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound can be prepared by modifying functional groups present in the active compound in such a way that the modifications are cleaved, either in routine manipulation or in vivo, to the parent active compound. Some examples of prodrugs include compounds wherein a hydroxy, amino or mercapto group is bonded to any group that, when the prodrug of the active compound is administered to a subject, the group cleaves to form a free hydroxy, free amino or free mercapto group, respectively. Some other examples of prodrugs include, but are not limited to, acetate, formate and benzoate derivatives of an alcohol or acetamide, formamide and benzamide derivatives of an amine functional group in the active compound and the like. See, for example, Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11,:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Prodrug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

The disclosure is further illustrated by the following examples which should not be construed as limiting. The examples are illustrative only, and are not intended to limit, in any manner, any of the aspects described herein. Further, various changes and modifications to the disclosed embodiments, which will be apparent to those of skill in the art, can be made without departing from the spirit and scope of the present invention. The following examples do not in any way limit the invention.

### Examples

The inventors have been testing pharmacologics that reduce adhesion for over 10 years in a well-established and characterized animal model. Recently, the inventors tested several currently approved drugs, and discovered that several types of histone deacetylase inhibitors are unexpectedly very effective in reducing adhesion formation in a single intraoperative dose.

Accordingly, the inventors conducted a proof of principle experiment and several follow-up studies with valproic acid (inhibits histones 1 and 2a), a class VIII HDAC inhibitor, using a previously described rodent model of adhesion formation and demonstrated efficacy in reducing intraabdominal adhesions in a single low dose (50 mg/kg) administration at surgery. Preoperative, intraoperative and postoperative dosing regimens were tested. The accompanying data further describes the methods, dosing and adhesion reduction results specific to valproic acid. Data for the results of these studies is shown in FIGS. 1A-3B.

As can be seen in FIG. 1A, valproic acid at 100 mg/kg administered intraperitoneally in 3 doses (24 hr pre-op, intra-op, 24 hrs post-op) significantly reduced adhesion formation at 7 days. As can be seen in FIG. 1B, valproic acid at 50 mg/kg administered intraperitoneally in 3 doses (24 hr pre-op, intra-op, 24 hrs post-op) significantly reduced adhesion formation at 7 days when administered at a dose of 50 mg/kg.

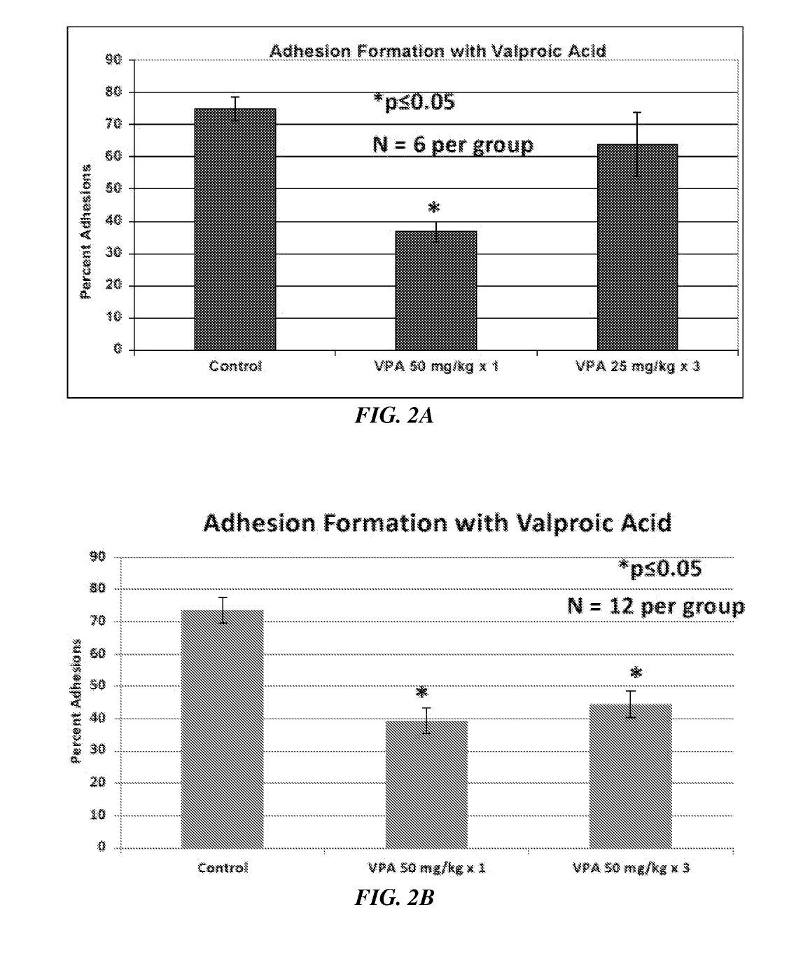

As seen in FIG. 2A, valproic acid at 50 mg/kg administered intraperitoneally in a single intra-op dose significantly reduced adhesion formation at 7 days as compared with three doses (pre-op, intra-op and pre-op) of 25 mg/kg of valproic acid. While both dosing regimens were effective, the single intra-operative dose of 50 mg/kg was superior to multiples doses of 25 mg/kg. As seen in FIG. 2B, valproic acid at 50 mg/kg administered intraperitoneally in 3 doses (24 hr pre-op, intra-op, 24 hrs post-op) significantly reduced adhesion formation at 7 days but was no more effective than a single intra-op dose. Without wishing to be bound by a theory, a single intra-operative dose can be preferable for its simplicity.

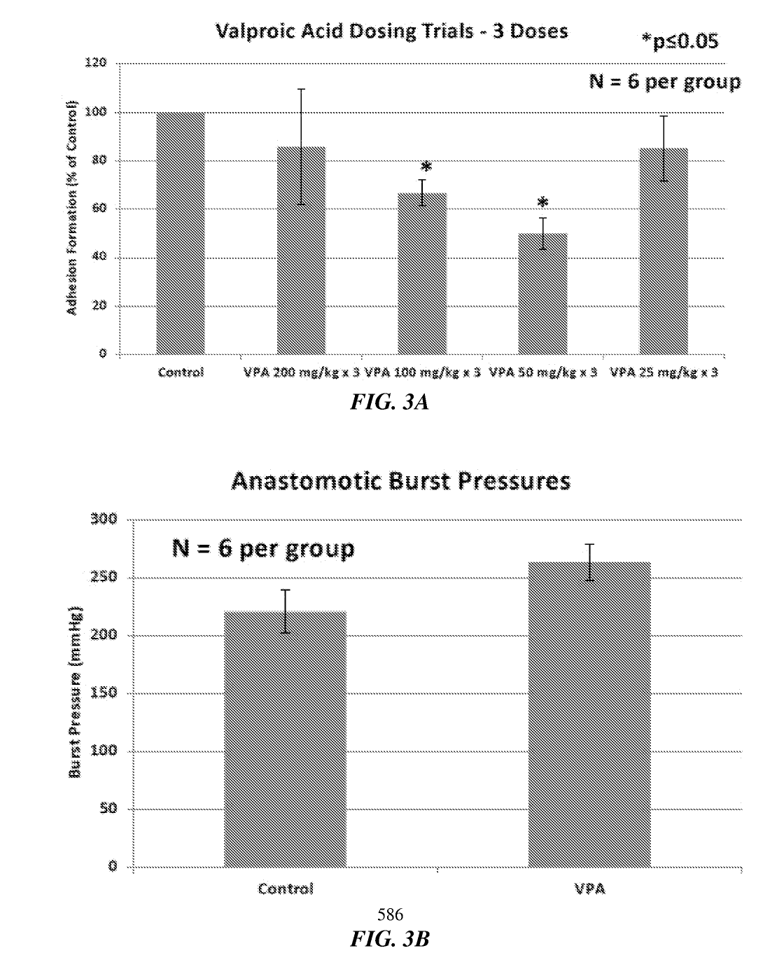

As can be seen in FIG. 3A, various amounts (200 mg/kg to 25 mg/kg) of valproic acid administered in three doses (pre-operation, intra-operation and post-operation) were all effective in reducing adhesion formation. However, the single intra-operative dose of 50 mg/kg was the most effective regimen. As can be seen in FIG. 3B, valproic acid at 50 mg/kg administered intraperitoneally in a single intra-op dose did not affect anastomotic wound healing at 7 days. This shows that there are no negative effects caused by the treatment with the HDAC inhibitor under the conditions tested.

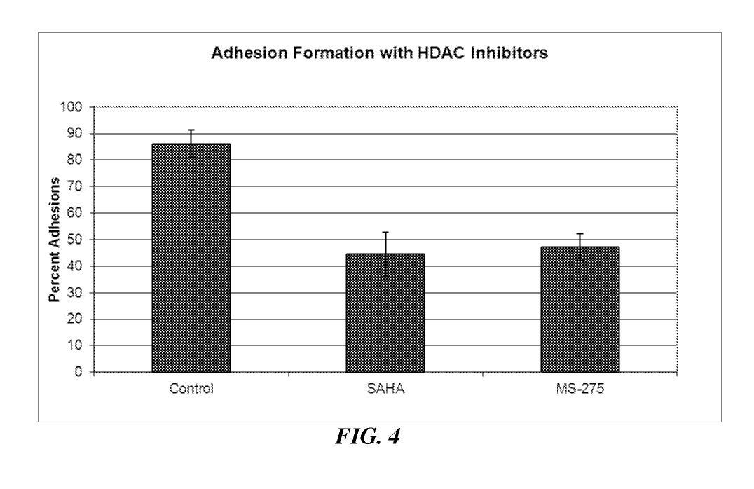

The inventors then tested two other HDAC inhibitors, specifically, SAHA (known as Verinostat (inhibits histones 1 and 3)) and MS-275 and MS-275 (known as Entinostat (inhibits histones 1, 2, 3, 4, and 8)). As seen in FIG. 4 and summarized in Table 1, both SAHA and MS-275 were also effective for the treatment of surgical adhesions when administered in a single intra-operative dose.

TABLE 1

| Group | Mean | N | SEM | SD | % Reduction |
|---|---|---|---|---|---|
| Control | 86.1111 | 6 | 5.121969 | 12.54621 | |
| SAHA | 44.4444 | 6 | 8.240221 | 20.18434 | 48.387097 |
| MS-275 | 47.2222 | 6 | 5.121969 | 12.54621 | 45.16129 |

The surgical procedure described in Chu et al. (Surgery, 2011, 149(6): 801-812), content of which is herein incorporated by reference in its entirety, was used to test the HDAC inhibitors discussed herein. More specifically, Male Wistar rats received 0.9% saline (Control) intraperitoneally in amounts specified in the figures on preoperative day 1, day of operation, and postoperative day 1. Adhesions were induced on the day of operation using our previously described ischemic button model. Animals were killed on postoperative day 7 for adhesion scoring. The effect of the HDAC inhibitors on intestinal wound healing was measured using colonic anastomotic burst pressures.

Animals:

Male Wistar rats (range, 200-225 g; Charles River Laboratories, Wilmington, Mass.) warehoused at constant room temperature and under 12-hour light/12-hour dark cycles and allowed access to food and water ad libitum. The Institutional Animal Care and Use Committee at the Boston University School of Medicine approved this study, and all procedures described were performed in accordance with recommendations outlined in the Guide for the Care and Use of Laboratory Animals: Eighth Edition (NRC 2011) published by the National Academies of Science.

Adhesion Operation:

Intraperitoneal ischemic buttons were created as described in Reed et al. (J. Surgical Res., 2002, 108:165-172), content of which is herein incorporated by reference in its entirety. In brief, after a midline laparotomy, 6 ischemic buttons (3 on each side) were created by ligating 5 mm of parietal peritoneum with 4-0 silk suture. Adhesion formation was quantified in a blinded fashion on postoperative day 7 by scoring the percentage of buttons with attached fibrinous adhesions in each rat. For example, an animal that had adhesions attached to 4 of its 6 ischemic buttons received a score of 66%.

Anastomotic Burst Pressure Measurements:

In the final experiment, the inventors tested the effects of intraperitoneal HDAC inhibitors on anastomotic wound healing. Male Wistar rats were randomized to operative controls (OP Control) or operative treatment with HDAC inhibitor as noted in the figure. Animals underwent the standard dosing regimen and received a standardized colonic anastomosis. In brief, after laparotomy and division of the distal colon, an end-to-end colonic anastomosis was created using a single running layer of 7-0 polypropylene suture. Animals were killed 7 days postoperatively and, after removing a 4-cm segment containing the anastomosis, the segment underwent measurements of burst pressure as described in Aarons et al. (Annals of Surgery, 2007, 245(2): 176-184), content of which is incorporated herein by reference in its entirety.

With reference to FIGS. 1A, 1B, 2A, 2B, 3A, 3B and 4, generally, the results suggest that there is an optimal dose of HDAC inhibitor that can be effective for the treatment of post surgical adhesions and that various HDAC inhibitors exhibit similar effects.

### REFERENCES

1. Aarons et al., “Statins (HMG-CoA reductase inhibitors) decrease postoperative adhesions by increasing peritoneal fibrinolytic activity”, Ann Surg, 245: 176-84 (2007).
2. Chu et al., “N-acetyl-L-cysteine decreases intra-abdominal adhesion formation through the upregulation of peritoneal fibrinolytic activity and antioxidant defenses”, Surgery, 149(6): 801-812 (June 2011).
3. Reed et al., “Neurokinin-1 receptor and substance P messenger RNA levels increase during intraabdominal adhesion formation”, J Surg Res; 108: 165-172 (2002).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1. A method of treating or inhibiting adhesion formation or reformation in a subject, the method comprising administering to a subject in need thereof a therapeutically effective amount of an histone deacetylase (HDAC) inhibitor.

2-31. (canceled)

32. The method of claim 1, wherein the HDAC inhibitor is administered directly into the peritoneal cavity to a site of the adhesion.

33. The method of claim 1, wherein said administration is in a single intraoperative dose.

34. The method of claim 1, wherein the HDAC inhibitor is administered in a soluble form, a gel, a spray, in a controlled-dose or sustained release formulation, in a liposome formulation, as a physical barrier, or as a lavage

35. The method of claim 1, wherein the HDAC inhibitor is applied to a biodegradable barrier that is placed in the subject during surgery.

36. The method of claim 1, wherein the HDAC inhibitor is administered topically at the site of an injury or surgery.

37. The method of claim 1, wherein the HDAC inhibitor is formulated into a composition which further comprises a pharmaceutically acceptable carrier.

38. The method of claim 1, wherein the subject has undergone or is undergoing surgery.

39. The method of claim 1, wherein the subject has a fibrotic disorder.

40. The method of claim 1, wherein the adhesion is a surgical adhesion or an intra-abdominal or peritoneal adhesion.

41. The method of claim 1, wherein the subject is a mammal.

42. The method of claim 1, the subject is a human.

43. The method of claim 1, wherein the HDAC inhibitor is selected from the group consisting of small organic or inorganic molecules, saccharines, oligosaccharides, polysaccharides, peptides, proteins, peptide analogs and derivatives, peptidomimetics, antibodies, functional portions of antibodies, nucleic acids, nucleic acid analogs and derivatives, an extract made from biological materials, naturally occurring or synthetic compositions, and any combinations thereof.

44. The method of claim 1, wherein the HDAC inhibitor is Valproic acid (VPA), SAHA, MS-275, or any combinations thereof.

45. The method of claim 1, further comprising administering a therapeutic agent to the subject.

46. The method of claim 1, wherein the HDAC inhibitor is administered to the subject during or following a surgical procedure.

47. The method of claim 46, wherein the surgical procedure comprises introducing an implant into the subject.

48. The method of claim 46, wherein the HDAC inhibitor is administered to the subject one or more times during the period of 1 hour-10 days prior to the surgical procedure.

49. The method of claim 46, wherein the HDAC inhibitor is administered to the subject one or more times during the surgical procedure.

50. The method of claim 46, wherein the HDAC inhibitor is administered to the subject one or more times during the period of 1 hour-10 days following the surgical procedure.

51. A method comprising contacting a subject with an HDAC inhibitor during surgery, wherein said contacting is performed by way of an intraperitoneal injection, thereby treating adhesion in the subject.
